(19) United States
(12) Patent Application Publication
(10) Pub. No.: US 2015/0110792 A1
(43) Pub. Date: Apr. 23, 2015

(54) ANTI-TIM-1 Antibodies And Uses Thereof

(71) Applicants: Biogen Idec MA Inc., Cambridge, MA (US); University of Iowa Research Foundation, Iowa City, IA (US)

(72) Inventors: Veronique Bailly, Boxborough, MA (US); Ellen Garber, Cambridge, MA (US); Paul D. Rennert, Holliston, MA (US); Nicholas Joseph Lennemann, Iowa City, IA (US); Wendy Jean Maury, Coralville, IA (US); Sven Henrik Moller-Tank, Iowa City, IA (US)

(21) Appl. No.: 14/359,784

(22) PCT Filed: Nov. 16, 2012
(86) PCT No.: PCT/US12/65530

§ 371 Date: May 21, 2014

Related U.S. Application Data
(60) Provisional application No. 61/562,684, filed on Nov. 22, 2011.

(51) Int. Cl. C07K 16/28

(52) U.S. Cl. CPC C07K 16/28; C07K 2317/56; C07K 2317/565; C07K 2317/76
USPC 424/139.1; 530/387.9; 530/387.3; 435/252.3; 435/375; 435/252.33; 435/254.23; 435/254.2; 435/254.21; 435/331

(57) ABSTRACT

Antibodies and antibody fragments that bind to human TIM-1 on the BED face of the protein are disclosed. Also disclosed are methods of using the antibodies and antibody fragments to inhibit or reduce TIM-1 binding to phosphatidylserine, inhibit or reduce TIM-1 binding to dendritic cells, and treat or prevent immunological disorders such as inflammatory and autoimmune conditions.

### STATEMENT REGARDING FEDERALLY SPONSORED RESEARCH OR DEVELOPMENT

This invention was made with government support under grants T32AI007533 and RO1 AI 077519 awarded by the National Institutes of Health. The government has certain rights in the invention.

### BACKGROUND

TIM-1, also known as HAVCR1 and KIM-1, has been identified as a susceptibility gene for human asthma (McIntire et al., 2003, Nature 425:576). TIM-1 is a type I membrane protein with an extracellular region containing an IgV domain, a mucin-rich domain, and a short membrane-proximal stalk containing N-linked glycosylation sites (Ichimura et al., 1998, J, Biol, Chem. 273(7):4135-42). The TIM-1 IgV domain has a disulfide-dependent conformation in which the CC′ loop is folded onto the GFC β strands, resulting in a distinctive cleft formed by the CC′ and FG loops (Santiago et al., 2007, Immunity 26(3):299-310). The cleft built by the CC′ and FG loops is a binding site for phosphatidylserine (Kobayashi et al., 2007, Immunity 27(6):927-40). Antibodies directed to the CC′/FG cleft of the TIM-1 IgV domain inhibit TIM-1 binding to phosphatidylserine and dendritic cells and exhibit therapeutic activity in vivo in a humanized mouse model of allergic asthma (Sonar et al., 2010, J. Clin. Invest. 120: 2767-81).

### SUMMARY

The invention is based, at least in part, on the identification and characterization of an antibody that binds to human TIM-1 on the BED face of the protein and inhibits TIM-1 binding to phosphatidylserine and dendritic cells and reduces symptoms of acute allergic asthma in a humanized animal model. Surprisingly, the anti-TIM-1 antibody mediates these functions even though it binds the receptor at an epitope located on a face of the IgV domain that is opposite that of the phosphatidylserine-interacting FG/CC′ cleft.

In one aspect, the invention features an isolated TIM-1-binding protein (e.g., an isolated antibody or antigen-binding fragment thereof) that selectively binds to the polypeptide of SEQ ID NO:1, when expressed on the surface of a cell, at an epitope that includes arginine amino acid residues at positions 85 and 86 of SEQ ID NO:1. The term “selectively binds” refers to binding of the TIM-1-binding protein to its target protein (e.g., the polypeptide of SEQ ID NO:1) in a manner that exhibits specificity to the target protein when present in a population of heterogeneous proteins (i.e., “selective” binding does not encompass non-specific protein-protein interactions).

As used herein, binding “at an epitope that includes arginine amino acid residues at positions 85 and 86 of SEQ ID NO:1” refers to the ability of an antibody or antigen-binding fragment thereof to selectively bind to the wild-type human TIM-1 protein of SEQ ID NO:1 but the inability to significantly bind to a mutant of SEQ ID NO:1 that contains an alanine substituted for arginine at position 85 and/or position 86 (i.e., wherein binding to a mutant of SEQ ID NO:1 that contains an alanine substituted for arginine at position 85 and/or position 86 occurs at a level that is less than 50% the level of binding that occurs to the wild-type human TIM-1 protein of SEQ ID NO:1 under the same assay conditions). In some embodiments, binding to a mutant of SEQ ID NO:1 that contains an alanine substituted for arginine at position 85 and/or position 86 occurs at a level that is less than 40%, less than 30%, less than 20%, less than 10%, or less than 5% the level of binding that occurs to the wild-type human TIM-1 protein of SEQ ID NO:1 under the same assay conditions.

Also disclosed is an isolated TIM-1-binding protein (e.g., an isolated antibody or antigen-binding fragment thereof) that selectively binds to the polypeptide of SEQ ID NO:1, when expressed on the surface of a cell, and crossblocks binding of the monoclonal antibody ARD5 to SEQ ID NO:1.

A TIM-1-binding protein crossblocks binding of a monoclonal antibody (e.g., ARD5) to TIM-1 when the TIM-1-binding protein's prior binding to TIM-1 inhibits later binding of the monoclonal antibody to TIM-1 at the same level at which the monoclonal antibody's prior binding to TIM-1 inhibits later binding of the identical monoclonal antibody to TIM-1. For example, a TIM-1-binding protein crossblocks binding of ARD5 to TIM-1 when the TIM-1-binding protein's prior binding to TIM-1 inhibits later binding of ARD5 to TIM-1 at the same level at which ARD5's prior binding to TIM-1 inhibits later binding of the identical monoclonal antibody to TIM-1. In certain embodiments, a TIM-1-binding protein crossblocks the binding of ARD5 to human TIM-1 to a level that is at least about 30%, 50%, 70%, 80%, 90%, 95%, 98% or 99% of crossblocking achieved by ARD5 of itself.

In certain embodiments, ARD5 crossblocks the binding of a TIM-1-binding protein to human TIM-1 to a level that is at least about 30%, 50%, 70%, 80%, 90%, 95%, 98% or 99% of crossblocking achieved by the TIM-1-binding protein of itself.

In certain embodiments, (i) a TIM-1-binding protein crossblocks the binding of ARD5 to human TIM-1 and (ii) ARD5 crossblocks the binding of the TIM-1-binding protein to human TIM-1. Complete crossblocking both ways indicates that the two TIM-1 binding proteins (e.g., antibodies) have the same footprint, i.e., bind to the same epitope. In certain embodiments, crossblocking one way or both ways is not complete, but partial, e.g., to a level that is at least about 30%, 50%, 70%, 80%, 90%, 95%, 98% or 99% of crossblocking achieved by the antibody itself. A partial crossblocking one way or both ways indicates that the footprints of the two antibodies are not identical, but may be overlapping or in close proximity.

Crossblocking experiments may be conducted with the test TIM-1-binding protein being present at or above saturating concentrations for TIM-1 binding based on its binding affinity.

In certain embodiments, a TIM-1-binding protein binds to the same epitope or substantially the same epitope as that of ARD5, as characterized by one or more of the experiments described herein, e.g., crossblocking experiments and the binding experiments to various TIM-1 species and mutated TIM-1 proteins.

Also disclosed is an isolated TIM-1-binding protein (e.g., an isolated antibody or antigen-binding fragment thereof) that selectively binds to the polypeptide of SEQ ID NO:1, when expressed on the surface of a cell, and crossblocks binding to SEQ ID NO:1 of a monoclonal antibody comprising the VH and VL domains of ARD5.

Also disclosed is an isolated TIM-1-binding protein (e.g., an isolated antibody or antigen-binding fragment thereof) that selectively binds to the polypeptide of SEQ ID NO:1, when expressed on the surface of a cell, at the same epitope as a monoclonal antibody comprising the VH and VL domains of ARD5.

In some embodiments, binding of a TIM-1-binding protein (e.g., an isolated antibody or antigen-binding fragment thereof) described herein to the polypeptide of SEQ ID NO:1 inhibits or reduces binding of TIM-1 to phosphatidylserine and/or dendritic cells. Binding may be decreased by a factor of at least about 10%, 30%, 50%, 70%, 80%, 90%, 95%, or 100%.

Further disclosed herein is an isolated TIM-1-binding protein (e.g., an isolated antibody or antigen-binding fragment thereof) that selectively binds to the polypeptide of SEQ ID NO:1, when expressed on the surface of a cell, and that also binds significantly (or detectably) to Cynomolgus TIM-1.

In certain embodiments, an anti-TIM-1 antibody binds substantially to the same epitope as that to which ARD5 binds. Whether two antibodies bind substantially to the same epitope can be determined by a competition assay. Such an assay may be conducted by labeling a control antibody (e.g., ARD5) with a detectable label, such as biotin. The intensity of the bound label to TIM-1 is measured. If the labeled antibody competes with the unlabeled (test antibody) by binding to an overlapping epitope, the intensity will be decreased relative to the binding by negative control unlabeled antibody.

Also disclosed is an isolated TIM-1-binding protein (e.g., an isolated antibody or antigen-binding fragment thereof) that (i) selectively binds to the polypeptide of SEQ ID NO:1, when expressed on the surface of a cell, and (ii) comprises a VH domain that is at least 80% identical to the amino acid sequence of SEQ ID NO:4. In some embodiments, the VH domain is at least 90% identical to the amino acid sequence of SEQ ID NO:4. In some embodiments, the VH domain is at least 95% identical to the amino acid sequence of SEQ ID NO:4. In some embodiments, the VH domain is identical to the amino acid sequence of SEQ ID NO:4.

Also disclosed is an isolated TIM-1-binding protein (e.g., an isolated antibody or antigen-binding fragment thereof) that (i) selectively binds to the polypeptide of SEQ ID NO:1, when expressed on the surface of a cell, and (ii) comprises a VL domain that is at least 80% identical to the amino acid sequence of SEQ ID NO:6. In some embodiments, the VL domain is at least 90% identical to the amino acid sequence of SEQ ID NO:6. In some embodiments, the VL domain is at least 95% identical to the amino acid sequence of SEQ ID NO:6. In some embodiments, the VL domain is identical to the amino acid sequence of SEQ ID NO:6.

Also disclosed is an isolated TIM-1-binding protein (e.g., an isolated antibody or antigen-binding fragment thereof) that (i) selectively binds to the polypeptide of SEQ ID NO:1, when expressed on the surface of a cell, (ii) comprises a VH domain that is at least 80% identical to the amino acid sequence of SEQ ID NO:4, and (iii) comprises a VL domain that is at least 80% identical to the amino acid sequence of SEQ ID NO:6. In some embodiments, (i) the VH domain is at least 90% identical to the amino acid sequence of SEQ ID NO:4, and (ii) the VL domain is at least 90% identical to the amino acid sequence of SEQ ID NO:6. In some embodiments, (i) the VH domain is at least 95% identical to the amino acid sequence of SEQ ID NO:4, and (ii) the VL domain is at least 95% identical to the amino acid sequence of SEQ ID NO:6. In some embodiments, (i) the VH domain is identical to the amino acid sequence of SEQ ID NO:4, and (ii) the VL domain is identical to the amino acid sequence of SEQ ID NO:6.

Also disclosed is an isolated TIM-1-binding protein (e.g., an isolated antibody or antigen-binding fragment thereof) that (i) selectively binds to the polypeptide of SEQ ID NO:1, when expressed on the surface of a cell, and (ii) comprises a VH domain comprising a first heavy chain complementarity determining region (CDR) that is at least 90% identical to CDR-H1 of SEQ ID NO:4, a second heavy chain CDR that is at least 90% identical to CDR-H2 of SEQ ID NO:4, and a third heavy chain CDR that is at least 90% identical to CDR-H3 of SEQ ID NO:4. In some embodiments, the first heavy chain CDR is at least 95% identical to CDR-H1 of SEQ ID NO:4, the second heavy chain CDR is at least 95% identical to CDR-H2 of SEQ ID NO:4, and the third heavy chain CDR is at least 95% identical to CDR-H3 of SEQ ID NO:4. In some embodiments, the first heavy chain CDR is identical to CDR-H1 of SEQ ID NO:4, the second heavy chain CDR is identical to CDR-H2 of SEQ ID NO:4, and the third heavy chain CDR is identical to CDR-H3 of SEQ ID NO:4.

Also disclosed is an isolated TIM-1-binding protein (e.g., an isolated antibody or antigen-binding fragment thereof) that (i) selectively binds to the polypeptide of SEQ ID NO:1, when expressed on the surface of a cell, and (ii) comprises a VL domain comprising a first light chain CDR that is at least 90% identical to CDR-L1 of SEQ ID NO:6, a second light chain CDR that is at least 90% identical to CDR-L2 of SEQ ID NO:6, and a third light chain CDR that is at least 90% identical to CDR-L3 of SEQ ID NO:6. In some embodiments, the first light chain CDR is at least 95% identical to CDR-L1 of SEQ ID NO:6, the second light chain CDR is at least 95% identical to CDR-L2 of SEQ ID NO:6, and the third light chain CDR is at least 95% identical to CDR-L3 of SEQ ID NO:6. In some embodiments, the first light chain CDR is identical to CDR-L1 of SEQ ID NO:6, the second light chain CDR is identical to CDR-L2 of SEQ ID NO:6, and the third light chain CDR is identical to CDR-L3 of SEQ ID NO:6.

Also disclosed is an isolated TIM-1-binding protein (e.g., an isolated antibody or antigen-binding fragment thereof) that (i) selectively binds to the polypeptide of SEQ ID NO:1, when expressed on the surface of a cell, (ii) comprises a VH domain comprising a first heavy chain CDR that is at least 90% identical to CDR-H1 of SEQ ID NO:4, a second heavy chain CDR that is at least 90% identical to CDR-H2 of SEQ ID NO:4, and a third heavy chain CDR that is at least 90% identical to CDR-H3 of SEQ ID NO:4, and (iii) comprises a VL domain comprising a first light chain CDR that is at least 90% identical to CDR-L1 of SEQ ID NO:6, a second light chain CDR that is at least 90% identical to CDR-L2 of SEQ ID NO:6, and a third light chain CDR that is at least 90% identical to CDR-L3 of SEQ ID NO:6. In some embodiments, (i) the first heavy chain CDR is at least 95% identical to CDR-H1 of SEQ ID NO:4, the second heavy chain CDR is at least 95% identical to CDR-H2 of SEQ ID NO:4, and the third heavy chain CDR is at least 95% identical to CDR-H3 of SEQ ID NO:4, and (ii) the first light chain CDR is at least 95% identical to CDR-L1 of SEQ ID NO:6, the second light chain CDR is at least 95% identical to CDR-L2 of SEQ ID NO:6, and the third light chain CDR is at least 95% identical to CDR-L3 of SEQ ID NO:6. In some embodiments, (i) the first heavy chain CDR is identical to CDR-H1 of SEQ ID NO:4, the second heavy chain CDR is identical to CDR-H2 of SEQ ID NO:4, and the third heavy chain CDR is identical to CDR-H3 of SEQ ID NO:4, and (ii) the first light chain CDR is identical to CDR-L1 of SEQ ID NO:6, the second light chain CDR is identical to CDR-L2 of SEQ ID NO:6, and the third light chain CDR is identical to CDR-L3 of SEQ ID NO:6.

An antibody or antigen-binding fragment thereof described herein can optionally contain framework regions that are collectively at least 90% identical (or at least 95, 98, or 99% identical) to human germline framework regions. The term “collectively” means that all frameworks are considered together in the sequence comparison, rather than individual framework regions. For example, an antibody or antigen-binding fragment thereof described herein can comprise VH domain framework regions that are collectively at least 90% identical (or at least 95, 98, or 99% identical) to the framework regions of the VH domain of SEQ ID NO:4. In another example, an antibody or antigen-binding fragment thereof described herein can comprise VL domain framework regions that are collectively at least 90% identical (or at least 95, 98, or 99% identical) to the framework regions of the VL domain of SEQ ID NO:6. In some cases, an antibody or antigen-binding fragment thereof described herein can comprise (i) VH domain framework regions that are collectively at least 90% identical to the framework regions of the VH domain of SEQ ID NO:4, and (ii) VL domain framework regions that are collectively at least 90% identical to the framework regions of the VL domain of SEQ ID NO:6.

Also disclosed is an isolated TIM-1-binding protein (e.g., an isolated antibody or antigen-binding fragment thereof) that (i) selectively binds to the polypeptide of SEQ ID NO:1, when expressed on the surface of a cell, (ii) comprises a VH domain comprising SEQ ID NO:4, and (iii) comprises a VL domain comprising SEQ ID NO:6.

Also disclosed is an isolated TIM-1-binding protein (e.g., an isolated antibody or antigen-binding fragment thereof) that (i) selectively binds to the polypeptide of SEQ ID NO:1, when expressed on the surface of a cell, (ii) comprises a VH domain comprising CDRs that are identical to the CDRs of SEQ ID NO:4 or wherein each CDR differs from the corresponding CDR of SEQ ID NO:4 in at most one, two, three, or four alterations (e.g., substitutions, deletions, or insertions), wherein the framework regions are collectively at least 90, 95, 97, 98, or 99% identical to the framework regions of SEQ ID NO:4, and (iii) comprises a VL domain comprising CDRs that are identical to the CDRs of SEQ ID NO:6 or wherein each CDR differs from the corresponding CDR of SEQ ID NO:6 in at most one, two, three, or four alterations (e.g., substitutions, deletions, or insertions), wherein the framework regions are collectively at least 90, 95, 97, 98, or 99% identical to the framework regions of SEQ ID NO:6.

In one embodiment, the antibody or antigen-binding fragment includes three or all six CDRs from ARD5 or closely related CDRs, e.g., CDRs that are identical or have at least one amino acid alteration, but not more than two, three or four alterations (e.g., substitutions, deletions, or insertions), or other CDR described herein.

An antibody or antigen-binding fragment described herein can be, for example, a humanized antibody, a fully human antibody, a monoclonal antibody, a single chain antibody, a monovalent antibody, a polyclonal antibody, a chimeric antibody, a multispecific antibody (e.g., a bispecific antibody), a multivalent antibody, an F_{ab} fragment, an F_{(ab′)2} fragment, an F_{ab′} fragment, an F_{sc} fragment, or an Fᵥ fragment.

An antibody or antigen-binding fragment described herein may be “multispecific,” e.g., bispecific, trispecific or of greater multispecificity, meaning that it recognizes and binds to two or more different epitopes present on one or more different antigens (e.g., proteins) at the same time. Thus, whether a binding molecule is “monospecfic” or “multispecific,” e.g., “bispecific,” refers to the number of different epitopes with which the binding molecule reacts. Multispecific antibodies may be specific for different epitopes of a TIM-1 protein, or may be specific for TIM-1 as well as for a heterologous epitope, such as a heterologous polypeptide or solid support material.

As used herein the term “valent” (as used in “multivalent antibody”) refers to the number of potential binding domains, e.g., antigen binding domains, present in a binding molecule. Each binding domain specifically binds one epitope. When a binding molecule comprises more than one binding domain, each binding domain may specifically bind the same epitope (for an antibody with two binding domains, termed “bivalent monospecific”) or to different epitopes (for an antibody with two binding domains, termed “bivalent bispecific”). An antibody may also be bispecific and bivalent for each specificity (termed “bispecific tetravalent antibodies”). In another embodiment, tetravalent minibodies or domain deleted antibodies can be made.

Bispecific bivalent antibodies, and methods of making them, are described, for instance in U.S. Pat. Nos. 5,731,168; 5,807,706; 5,821,333; and U.S. Application Publication Nos. 2003/020734 and 2002/0155537, the disclosures of all of which are incorporated by reference herein. Bispecific tetravalent antibodies, and methods of making them are described, for instance, in WO 02/096948 and WO 00/44788, the disclosures of both of which are incorporated by reference herein. See generally, PCT publications WO 93/17715; WO 92/08802; WO 91/00360; WO 92/05793; WO 2007/109254; Tutt et al., J. Immunol. 147:60-69 (1991); U.S. Pat. Nos. 4,474,893; 4,714,681; 4,925,648; 5,573,920; 5,601,819; Kostelny et al., J. Immunol. 148:1547-1553 (1992). These references are all incorporated by reference herein.

In certain embodiments, an anti-TIM-1 antibody, e.g., one or the two heavy chains of the antibody, is linked to one or more scFv to form a bispecific antibody. In other embodiments, an anti-TIM-1 antibody is in the form of an scFv that is linked to an antibody to form a bispecific molecule. Antibody-scFv constructs are described, e.g., in WO 2007/109254.

The heavy and light chains of the antibody can be substantially full-length. The protein can include at least one, and optionally two, complete heavy chains, and at least one, and optionally two, complete light chains or can include an antigen-binding fragment. In yet other embodiments, the antibody has a heavy chain constant region chosen from, e.g., IgG1, IgG2, IgG3, IgG4, IgM, IgA1, IgA2, IgD, and IgE. Typically, the heavy chain constant region is human or a modified form of a human constant region. In another embodiment, the antibody has a light chain constant region chosen from, e.g., kappa or lambda, particularly, kappa (e.g., human kappa).

Also provided herein are nucleic acids, e.g., DNAs, encoding an antibody or antigen binding fragment thereof described herein. Nucleic acids that are at least about 80%, 85%, 90%, 95%, 97%, 98% or 99% identical to or hybridize under stringent hybridization conditions to these nucleic acids are also encompassed herein.

Also disclosed is an isolated cell that produces an antibody or antigen-binding fragment described herein. Also provided herein are cells, e.g., isolated cells, comprising a nucleic acid encoding a protein described herein. The cell can be, for example, a fused cell obtained by fusing a mammalian B cell and myeloma cell.

Also disclosed is a pharmaceutical composition comprising an antibody or antigen-binding fragment described herein and a pharmaceutically acceptable carrier.

In another aspect, the invention features a method of inhibiting or reducing binding of TIM-1 to phosphatidylserine, the method comprising contacting a first cell that expresses TIM-1 with an amount of an antibody or antigen-binding fragment described herein effective to inhibit or reduce binding of the first cell to a second cell that contains phosphatidylserine on its cell surface.

Also disclosed is a method of inhibiting or reducing binding of TIM-1 to a dendritic cell, the method comprising contacting a cell that expresses TIM-1 with an amount of an antibody or antigen-binding fragment described herein effective to inhibit or reduce binding of the cell to a dendritic cell.

Also disclosed is a method of treating or preventing an inflammatory or autoimmune condition, the method comprising administering to a mammal having an inflammatory or autoimmune condition a pharmaceutical composition comprising a therapeutically effective amount of an antibody or antigen-binding fragment described herein.

Also disclosed is a method of treating or preventing asthma, the method comprising administering to a mammal having asthma a pharmaceutical composition comprising a therapeutically effective amount of an antibody or antigen-binding fragment described herein.

Also disclosed is a method of treating or preventing an atopic disorder, the method comprising administering to a mammal having an atopic disorder a pharmaceutical composition comprising a therapeutically effective amount of an antibody or antigen-binding fragment described herein. The atopic disorder can be, for example, atopic dermatitis, contact dermatitis, urticaria, allergic rhinitis, angioedema, latex allergy, or an allergic lung disorder (e.g., asthma, allergic bronchopulmonary aspergillosis, or hypersensitivity pneumonitis).

The mammal treated according to the methods described herein can be, e.g., a human, a mouse, a rat, a cow, a pig, a dog, a cat, or a monkey.

It should be understood that where reference is made herein to an “antibody or antigen-binding fragment,” this phrase may be replaced with “protein.” Accordingly, the description of the antibodies and antibody-binding fragments thereof also applies to proteins, such as proteins comprising these antibodies or antibody-binding fragments thereof.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, the exemplary methods and materials are described below. All publications, patent applications, patents, and other references mentioned herein are incorporated by reference in their entirety. In case of conflict, the present application, including definitions, will control. The materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a graph showing the ability of four different anti-TIM-1 antibodies to bind to human TIM-1 mutants (binding is shown as relative to the same antibody's binding to wild-type human TIM-1).

FIG. 2 is a depiction of the crystal structure of the murine TIM-1 protein (the amino acids that correspond to Arg85 and Arg86 of human TIM-1 lie in the BED face of the protein).

FIG. 3 is a planar alignment of two regions of the TIM-1 protein, which has been rotated to bring the BED face of the molecule forward. Region A contains the ARD5 epitope and Region B includes the N-linked glycosylation site present in the human TIM-1 protein.

FIG. 4 is a representation of the human TIM-1 amino acid sequence modeled (“threaded”) onto the murine TIM-1 crystal structure. The location of Arg85 and Arg86 in the model is darkened.

FIG. 5 is a graph depicting the effect of anti-TIM-1 antibodies on TIM-1 binding to phosphatidylserine.

FIG. 6 is a graph depicting the effect of the ARD5 monoclonal antibody on TIM-1 binding to dendritic cells.

FIGS. 7A and 7B are graphs of flow cytometric analysis depicting binding of anti-TIM-1 antibodies to JUN2 cells (FIG. 7A) and 769P cells (FIG. 7B).

FIG. 8 is a series of graphs of depicting an extremely fast on-rate and slow off-rate of ARD5 binding to human TIM-1 in a surface plasmon resonance assay.

FIG. 9 is a graph of FACS analysis depicting binding of anti-TIM-1 antibodies to 293 cells transfected with a human or Cynomolgus monkey TIM-1 cDNA.

FIG. 10 is a graph of FACS analysis depicting binding of anti-TIM-1 antibodies to an African Green Monkey cell line expressing TIM-1.

FIG. 11 is a series of histograms of FACS analyses depicting the effects of treatment with anti-TIM-1 antibodies on antigen-specific IgE production in Der p1-challenged mice humanized with peripheral blood mononuclear cells (PBMCs) from moderate to severe dust mite allergic asthmatics.

FIGS. 12A and 12B are graphs depicting IL-13 (FIG. 12A) and IL-4 (FIG. 12B) production by antigen-restimulated mononuclear cells isolated from the spleen of humanized SCID mice treated with anti-TIM-1 antibodies.

FIG. 13 is a graph depicting cell proliferation in response to Der p1 stimulation in mice treated with anti-TIM-1 antibodies. Restimulation was with 500 ng/ml Der p1 and the readout was after 24 hours. For each group, relative proliferation is expressed as (restimulated/medium only)*100 values.

FIG. 14 is a graph depicting the effect of treatment with the ARD5 monoclonal antibody on airway hyperreactivity in Der p1-challenged mice humanized with PBMC from moderate to severe dust mite allergic asthmatics.

### DETAILED DESCRIPTION

ARD5 is an exemplary monoclonal antibody that specifically binds to human TIM-1 on the BED face of the protein at an epitope that includes the arginine amino acid residues at positions 85 and 86. The anti-TIM-1 antibodies described herein inhibit TIM-1 binding to phosphatidylserine and dendritic cells and can be used to treat or prevent immunological disorders such as inflammatory and autoimmune conditions.

### TIM-1

The amino acid sequence of the human TIM-1 protein is shown as:

(SEQ ID NO: 1)
MHPQVVILSLILHLADSVAGSVKVGGEAGPSVTLPCHYSGAVTSMCW
NRGSCSLFTCQNGIVWTNGTHVTYRKDTRYKLLGDLSRRDVSLTIEN
TAVSDSGVYCCRVEHRGWFNDMKITVSLEIVPPKVTTTPIVTTVPTV
TTVRTSTTVPTTTTVPMTTVPTTTVPTTMSIPTTTTVLTTMTVSTTT
SVPTTTSIPTTTSVPVTTTVSTFVPPMPLPRQNHEPVATSPSSPQPA
ETHPTTLQGAIRREPTSSPLYSYTTDGNDTVTESSDGLWNNNQTQLF
LEHSLLTANTTKGIYAGVCISVLVLLALLGVIIAKKYFFKKEVQQLS
VSFSSLQIKALQNAVEKEVQAEDNIYIENSLYATD.

This human TIM-1 protein can be used as an immunogen to prepare anti-human TIM-1 antibodies. Anti-human TIM-1 antibodies can then be screened to identify antibodies having the features described herein (e.g., binding at an epitope that includes arginine amino acid residues at positions 85 and 86 of TIM-1).

### Anti-TIM-1 Antibodies

This disclosure includes the sequences of a specific monoclonal antibody, ARD5, that binds to human TIM-1 on the BED face of the protein at an epitope that includes the arginine amino acid residues at positions 85 and 86. Antibodies, such as ARD5, can be made, for example, by preparing and expressing synthetic genes that encode the recited amino acid sequences or by mutating human germline genes to provide a gene that encodes the recited amino acid sequences. Moreover, this antibody and other anti-TIM-1 antibodies can be produced, e.g., using one or more of the following methods.

Numerous methods are available for obtaining antibodies, particularly human antibodies. One exemplary method includes screening protein expression libraries, e.g., phage or ribosome display libraries. Phage display is described, for example, U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; and WO 90/02809. The display of Fab′s on phage is described, e.g., in U.S. Pat. Nos. 5,658,727; 5,667,988; and 5,885,793.

In addition to the use of display libraries, other methods can be used to obtain a TIM-1-binding antibody. For example, the TIM-1 protein or a peptide thereof can be used as an antigen in a non-human animal, e.g., a rodent, e.g., a mouse, hamster, or rat. In addition, cells transfected with a cDNA encoding TIM-1 can be injected into a non-human animal as a means of producing antibodies that effectively bind the cell surface TIM-1 protein.

In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XENOMOUSE™, Green et al. (1994) Nature Genetics 7:13-21, U.S. 2003-0070185, WO 96/34096, and WO 96/33735.

In another embodiment, a monoclonal antibody is obtained from the non-human animal, and then modified, e.g., humanized or deimmunized. Winter describes an exemplary CDR-grafting method that may be used to prepare humanized antibodies described herein (U.S. Pat. No. 5,225,539). All or some of the CDRs of a particular human antibody may be replaced with at least a portion of a non-human antibody. It may only be necessary to replace the CDRs required for binding or binding determinants of such CDRs to arrive at a useful humanized antibody that binds to TIM-1.

Humanized antibodies can be generated by replacing sequences of the Fv variable region that are not directly involved in antigen binding with equivalent sequences from human Fv variable regions. General methods for generating humanized antibodies are provided by Morrison, S. L. (1985) Science 229:1202-1207, by Oi et al. (1986) BioTechniques 4:214, and by U.S. Pat. No. 5,585,089; U.S. Pat. No. 5,693,761; U.S. Pat. No. 5,693,762; U.S. Pat. No. 5,859,205; and U.S. Pat. No. 6,407,213. Those methods include isolating, manipulating, and expressing the nucleic acid sequences that encode all or part of immunoglobulin Fv variable regions from at least one of a heavy or light chain. Sources of such nucleic acid are well known to those skilled in the art and, for example, may be obtained from a hybridoma producing an antibody against a predetermined target, as described above, from germline immunoglobulin genes, or from synthetic constructs. The recombinant DNA encoding the humanized antibody can then be cloned into an appropriate expression vector.

Human germline sequences, for example, are disclosed in Tomlinson, I. A. et al. (1992) J. Mol. Biol. 227:776-798; Cook, G. P. et al. (1995) Immunol. Today 16: 237-242; Chothia, D. et al. (1992) J. Mol. Bio. 227:799-817; and Tomlinson et al. (1995) EMBO J. 14:4628-4638. The V BASE directory provides a comprehensive directory of human immunoglobulin variable region sequences (compiled by Tomlinson, I. A. et al. MRC Centre for Protein Engineering, Cambridge, UK). These sequences can be used as a source of human sequence, e.g., for framework regions and CDRs. Consensus human framework regions can also be used, e.g., as described in U.S. Pat. No. 6,300,064.

A non-human TIM-1-binding antibody may also be modified by specific deletion of human T cell epitopes or “deimmunization” by the methods disclosed in WO 98/52976 and WO 00/34317. Briefly, the heavy and light chain variable regions of an antibody can be analyzed for peptides that bind to MHC Class II; these peptides represent potential T-cell epitopes (as defined in WO 98/52976 and WO 00/34317). For detection of potential T-cell epitopes, a computer modeling approach termed “peptide threading” can be applied, and in addition a database of human MHC class II binding peptides can be searched for motifs present in the V_{H} and V_{L} sequences, as described in WO 98/52976 and WO 00/34317. These motifs bind to any of the 18 major MHC class II DR allotypes, and thus constitute potential T cell epitopes. Potential T-cell epitopes detected can be eliminated by substituting small numbers of amino acid residues in the variable regions, or preferably, by single amino acid substitutions. As far as possible, conservative substitutions are made. Often, but not exclusively, an amino acid common to a position in human germline antibody sequences may be used. After the deimmunizing changes are identified, nucleic acids encoding V_{H} and V_{L} can be constructed by mutagenesis or other synthetic methods (e.g., de novo synthesis, cassette replacement, and so forth). A mutagenized variable sequence can, optionally, be fused to a human constant region, e.g., human IgG1 or kappa constant regions.

In some cases, a potential T cell epitope will include residues known or predicted to be important for antibody function. For example, potential T cell epitopes are usually biased towards the CDRs. In addition, potential T cell epitopes can occur in framework residues important for antibody structure and binding. Changes to eliminate these potential epitopes will in some cases require more scrutiny, e.g., by making and testing chains with and without the change. Where possible, potential T cell epitopes that overlap the CDRs can be eliminated by substitutions outside the CDRs. In some cases, an alteration within a CDR is the only option, and thus variants with and without this substitution can be tested. In other cases, the substitution required to remove a potential T cell epitope is at a residue position within the framework that might be critical for antibody binding. In these cases, variants with and without this substitution are tested. Thus, in some cases several variant deimmunized heavy and light chain variable regions are designed and various heavy/light chain combinations are tested to identify the optimal deimmunized antibody. The choice of the final deimmunized antibody can then be made by considering the binding affinity of the different variants in conjunction with the extent of deimmunization, particularly, the number of potential T cell epitopes remaining in the variable region. Deimmunization can be used to modify any antibody, e.g., an antibody that includes a non-human sequence, e.g., a synthetic antibody, a murine antibody other non-human monoclonal antibody, or an antibody isolated from a display library.

Other methods for humanizing antibodies can also be used. For example, other methods can account for the three dimensional structure of the antibody, framework positions that are in three dimensional proximity to binding determinants, and immunogenic peptide sequences. See, e.g., WO 90/07861; U.S. Pat. Nos. 5,693,762; 5,693,761; 5,585,089; 5,530,101; and 6,407,213; Tempest et al. (1991) Biotechnology 9:266-271. Still another method is termed “humaneering” and is described, for example, in U.S. 2005-008625.

The antibody can include a human Fc region, e.g., a wild-type Fc region or an Fc region that includes one or more alterations. In one embodiment, the constant region is altered, e.g., mutated, to modify the properties of the antibody (e.g., to increase or decrease one or more of: Fc receptor binding, antibody glycosylation, the number of cysteine residues, effector cell function, or complement function). For example, the human IgG1 constant region can be mutated at one or more residues, e.g., one or more of residues 234 and 237. Antibodies may have mutations in the CH2 region of the heavy chain that reduce or alter effector function, e.g., Fc receptor binding and complement activation. For example, antibodies may have mutations such as those described in U.S. Pat. Nos. 5,624,821 and 5,648,260. Antibodies may also have mutations that stabilize the disulfide bond between the two heavy chains of an immunoglobulin, such as mutations in the hinge region of IgG4, as disclosed in the art (e.g., Angal et al. (1993) Mol. Immunol. 30:105-08). See also, e.g., U.S. 2005-0037000.

### Affinity Maturation

In one embodiment, an anti-TIM-1 antibody is modified, e.g., by mutagenesis, to provide a pool of modified antibodies. The modified antibodies are then evaluated to identify one or more antibodies having altered functional properties (e.g., improved binding, improved stability, reduced antigenicity, or increased stability in vivo). In one implementation, display library technology is used to select or screen the pool of modified antibodies. Higher affinity antibodies are then identified from the second library, e.g., by using higher stringency or more competitive binding and washing conditions. Other screening techniques can also be used.

In some implementations, the mutagenesis is targeted to regions known or likely to be at the binding interface. If, for example, the identified binding proteins are antibodies, then mutagenesis can be directed to the CDR regions of the heavy or light chains as described herein. Further, mutagenesis can be directed to framework regions near or adjacent to the CDRs, e.g., framework regions, particularly within 10, 5, or 3 amino acids of a CDR junction. In the case of antibodies, mutagenesis can also be limited to one or a few of the CDRs, e.g., to make step-wise improvements.

In one embodiment, mutagenesis is used to make an antibody more similar to one or more germline sequences. One exemplary germlining method can include: identifying one or more germline sequences that are similar (e.g., most similar in a particular database) to the sequence of the isolated antibody. Then mutations (at the amino acid level) can be made in the isolated antibody, either incrementally, in combination, or both. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In one embodiment, as many germline residues are introduced into an isolated antibody as possible.

In one embodiment, mutagenesis is used to substitute or insert one or more germline residues into a CDR region. For example, the germline CDR residue can be from a germline sequence that is similar (e.g., most similar) to the variable region being modified. After mutagenesis, activity (e.g., binding or other functional activity) of the antibody can be evaluated to determine if the germline residue or residues are tolerated. Similar mutagenesis can be performed in the framework regions.

Selecting a germline sequence can be performed in different ways. For example, a germline sequence can be selected if it meets a predetermined criteria for selectivity or similarity, e.g., at least a certain percentage identity, e.g., at least 75, 80, 85, 90, 91, 92, 93, 94, 95, 96, 97, 98, 99, or 99.5% identity, relative to the donor non-human antibody. The selection can be performed using at least 2, 3, 5, or 10 germline sequences. In the case of CDR1 and CDR2, identifying a similar germline sequence can include selecting one such sequence. In the case of CDR3, identifying a similar germline sequence can include selecting one such sequence, but may include using two germline sequences that separately contribute to the amino-terminal portion and the carboxy-terminal portion. In other implementations, more than one or two germline sequences are used, e.g., to form a consensus sequence.

Calculations of “sequence identity” between two sequences are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences.

In other embodiments, the antibody may be modified to have an altered glycosylation pattern (i.e., altered from the original or native glycosylation pattern). As used in this context, “altered” means having one or more carbohydrate moieties deleted, and/or having one or more glycosylation sites added to the original antibody. Addition of glycosylation sites to the presently disclosed antibodies may be accomplished by altering the amino acid sequence to contain glycosylation site consensus sequences; such techniques are well known in the art. Another means of increasing the number of carbohydrate moieties on the antibodies is by chemical or enzymatic coupling of glycosides to the amino acid residues of the antibody. These methods are described in, e.g., WO 87/05330, and Aplin and Wriston (1981) CRC Crit. Rev. Biochem. 22:259-306. Removal of any carbohydrate moieties present on the antibodies may be accomplished chemically or enzymatically as described in the art (Hakimuddin et al. (1987) Arch. Biochem. Biophys. 259:52; Edge et al. (1981) Anal. Biochem. 118:131; and Thotakura et al. (1987) Meth. Enzymol. 138:350). See, e.g., U.S. Pat. No. 5,869,046 for a modification that increases in vivo half life by providing a salvage receptor binding epitope.

In one embodiment, an antibody has CDR sequences that differ only insubstantially from those of the ARD5 monoclonal antibody. Insubstantial differences include minor amino acid changes, such as substitutions of 1 or 2 out of any of typically 5-7 amino acids in the sequence of a CDR, e.g., a Chothia or Kabat CDR. Typically an amino acid is substituted by a related amino acid having similar charge, hydrophobic, or stereochemical characteristics. Such substitutions would be within the ordinary skills of an artisan. Unlike in CDRs, more substantial changes in structure framework regions (FRs) can be made without adversely affecting the binding properties of an antibody. Changes to FRs include, but are not limited to, humanizing a nonhuman-derived framework or engineering certain framework residues that are important for antigen contact or for stabilizing the binding site, e.g., changing the class or subclass of the constant region, changing specific amino acid residues which might alter an effector function such as Fc receptor binding (Lund et al. (1991) J. Immun. 147:2657-62; Morgan et al. (1995) Immunology 86:319-24), or changing the species from which the constant region is derived.

The anti-TIM-1 antibodies can be in the form of full length antibodies, or in the form of fragments of antibodies, e.g., Fab, F(ab′)₂, Fd, dAb, and scFv fragments. A fragment of an antibody can be an antigen-binding fragment, such as a variable region, e.g., VH or VL. Additional forms include a protein that includes a single variable domain, e.g., a camel or camelized domain. See, e.g., U.S. 2005-0079574 and Davies et al. (1996) Protein Eng. 9(6):531-7.

Provided herein are compositions comprising a mixture of an anti-TIM-1 antibody and one or more acidic variants thereof, e.g., wherein the amount of acidic variant(s) is less than about 80%, 70%, 60%, 60%, 50%, 40%, 30%, 30%, 20%, 10%, 5% or 1%. Also provided are compositions comprising an anti-TIM-1 antibody comprising at least one deamidation site, wherein the pH of the composition is from about 5.0 to about 6.5, such that, e.g., at least about 90% of the anti-TIM-1 antibodies are not deamidated (i.e., less than about 10% of the antibodies are deamidated). In certain embodiments, less than about 5%, 3%, 2% or 1% of the antibodies are deamidated. The pH may be from 5.0 to 6.0, such as 5.5 or 6.0. In certain embodiments, the pH of the composition is 5.5, 5.6, 5.7, 5.8, 5.9, 6.0, 6.1, 6.2, 6.3, 6.4 or 6.5.

An “acidic variant” is a variant of a polypeptide of interest which is more acidic (e.g. as determined by cation exchange chromatography) than the polypeptide of interest. An example of an acidic variant is a deamidated variant.

A “deamidated” variant of a polypeptide molecule is a polypeptide wherein one or more asparagine residue(s) of the original polypeptide have been converted to aspartate, i.e. the neutral amide side chain has been converted to a residue with an overall acidic character.

The term “mixture” as used herein in reference to a composition comprising an anti-TIM-1 antibody, means the presence of both the desired anti-TIM-1 antibody and one or more acidic variants thereof. The acidic variants may comprise predominantly deamidated anti-TIM-1 antibody, with minor amounts of other acidic variant(s).

In certain embodiments, the binding affinity (K_{D}), on-rate (K_{D} on) and/or off-rate (K_{D} off) of the antibody that was mutated to eliminate deamidation is similar to that of the wild-type antibody, e.g., having a difference of less than about 5 fold, 2 fold, 1 fold (100%), 50%, 30%, 20%, 10%, 5%, 3%, 2% or 1%.

In certain embodiments, an anti-TIM-1 antibody inhibits or reduces binding of TIM-1 to phosphatidylserine, inhibits or reduces binding of TIM-1 to dendritic cells, and/or reduces the severity of symptoms when administered in a humanized mouse model of acute allergic asthma. These features of an anti-TIM-1 antibody can be measured according to the methods described in the Examples.

### Antibody Fragments

Traditionally, antibody fragments were derived via proteolytic digestion of intact antibodies. Alternatively, these fragments can be produced directly by recombinant host cells. Fab, Fv and ScFv antibody fragments can all be expressed in and secreted from E. coli, thus allowing the facile production of large amounts of these fragments. Antibody fragments can be isolated from the antibody phage libraries. Alternatively, Fab′-SH fragments can be directly recovered from E. coli and chemically coupled to form F(ab)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. In other embodiments, the antibody of choice is a single chain Fv fragment (scFv). Fv and scFv contain intact combining sites that are devoid of constant regions; thus, they are suitable for reduced nonspecific binding during in vivo use. scFv fusion proteins may be constructed to yield fusion of an effector protein at either the amino or the carboxy terminus of an scFv. The antibody fragment may also be a “linear antibody,” e.g., as described in U.S. Pat. No. 5,641,870. Such linear antibody fragments may be monospecific or bispecific.

### Bispecific Antibodies

Bispecific antibodies are antibodies that have binding specificities for at least two different epitopes. Exemplary bispecific antibodies may bind to two different epitopes of the TIM-1 protein. Other such antibodies may combine a TIM-1 binding site with a binding site for another protein. Bispecific antibodies can be prepared as full length antibodies or antibody fragments (e.g., F(ab′)₂ bispecific antibodies).

Traditional production of full length bispecific antibodies is based on the co-expression of two immunoglobulin heavy chain-light chain pairs, where the two chains have different specificities (Millstein et al., Nature 305:537-539 (1983)). In a different approach, antibody variable domains with the desired binding specificities are fused to immunoglobulin constant domain sequences. DNAs encoding the immunoglobulin heavy chain fusions and, if desired, the immunoglobulin light chain, are inserted into separate expression vectors, and are co-transfected into a suitable host cell. This provides for greater flexibility in adjusting the proportions of the three polypeptide fragments. It is, however, possible to insert the coding sequences for two or all three polypeptide chains into a single expression vector when the expression of at least two polypeptide chains in equal ratios results in high yields.

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Heteroconjugate antibodies may be made using any convenient cross-linking methods.

The “diabody” technology provides an alternative mechanism for making bispecific antibody fragments. The fragments comprise a V_{H} connected to a V_{L} by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites.

### Multivalent Antibodies

A multivalent antibody may be internalized (and/or catabolized) faster than a bivalent antibody by a cell expressing an antigen to which the antibodies bind. The antibodies describe herein can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. An exemplary dimerization domain comprises (or consists of) an Fc region or a hinge region. A multivalent antibody can comprise (or consist of) three to about eight (e.g., four) antigen binding sites. The multivalent antibody optionally comprises at least one polypeptide chain (e.g., at least two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is a polypeptide chain of an Fc region, X1 and X2 represent an amino acid or peptide spacer, and n is 0 or 1.

### Antibody Production

Some antibodies, e.g., Fab′s, can be produced in bacterial cells, e.g., E. coli cells. Antibodies can also be produced in eukaryotic cells. In one embodiment, the antibodies (e.g., scFv's) are expressed in a yeast cell such as Pichia (see, e.g., Powers et al. (2001) J Immunol Methods. 251:123-35), Hanseula, or Saccharomyces.

In one preferred embodiment, antibodies are produced in mammalian cells. Exemplary mammalian host cells for expressing an antibody include Chinese Hamster Ovary (CHO cells) (including dhfr⁻ CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells and SP2 cells, COS cells, and a cell from a transgenic animal, e.g., a transgenic mammal. For example, the cell is a mammary epithelial cell.

In addition to the nucleic acid sequence encoding the diversified immunoglobulin domain, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin, or methotrexate, on a host cell into which the vector has been introduced.

In an exemplary system for antibody expression, a recombinant expression vector encoding both the antibody heavy chain and the antibody light chain is introduced into dhfr⁻ CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and the antibody is recovered from the culture medium. Standard molecular biology techniques are used to prepare the recombinant expression vector, transfect the host cells, select for transformants, culture the host cells and recover the antibody from the culture medium. For example, some antibodies can be isolated by affinity chromatography with a Protein A or Protein G coupled matrix.

Antibodies can also be produced by a transgenic animal. For example, U.S. Pat. No. 5,849,992 describes a method of expressing an antibody in the mammary gland of a transgenic mammal A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly. Animals are also provided comprising one or more of the nucleic acids described herein.

### Characterization

The binding properties of an antibody may be measured by any standard method, e.g., one of the following methods: BIACORE™ analysis, Enzyme Linked Immunosorbent Assay (ELISA), Fluorescence Resonance Energy Transfer (FRET), x-ray crystallography, sequence analysis and scanning mutagenesis.

### Surface Plasmon Resonance (SPR)

The binding interaction of a protein of interest and a target (e.g., TIM-1) can be analyzed using SPR. SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden). Information from SPR can be used to provide an accurate and quantitative measure of the equilibrium dissociation constant (K_{d}), and kinetic parameters, including Kₒₙ and K_{off}, for the binding of a biomolecule to a target.

Epitopes can also be directly mapped by assessing the ability of different antibodies to compete with each other for binding to human TIM-1 using BIACORE chromatographic techniques (Pharmacia BlAtechnology Handbook, “Epitope Mapping”, Section 6.3.2, (May 1994); see also Johne et al. (1993) J. Immunol. Methods, 160:191-198). Additional general guidance for evaluating antibodies, e.g., in Western blots and immunoprecipitation assays, can be found in Antibodies: A Laboratory Manual, ed. by Harlow and Lane, Cold Spring Harbor press (1988)).

### Deposits

The hybridoma producing the monoclonal antibody ARD5.12 (ARD5) has been deposited with the American Type Culture Collection (ATCC) under the terms of the Budapest Treaty on the International Recognition of the Deposit of Microorganisms for the Purpose of Patent Procedure on Oct. 26, 2011, and bears the accession number PTA-12195. Applicants acknowledge their duty to replace the deposit should the depository be unable to furnish a sample when requested due to the condition of the deposit before the end of the term of a patent issued hereon. Applicants also acknowledge their responsibility to notify the ATCC of the issuance of such a patent, at which time the deposit will be made available to the public. Prior to that time, the deposit will be made available to the Commissioner of Patents under the terms of 37 C.F.R. §1.14 and 35 U.S.C. §112.

Antibodies with Reduced Effector Function

The interaction of antibodies and antibody-antigen complexes with cells of the immune system triggers a variety of responses, referred to herein as effector functions. IgG antibodies activate effector pathways of the immune system by binding to members of the family of cell surface Fcγ receptors and to C1q of the complement system. Ligation of effector proteins by clustered antibodies triggers a variety of responses, including release of inflammatory cytokines, regulation of antigen production, endocytosis, and cell killing. In some clinical applications these responses are crucial for the efficacy of a monoclonal antibody. In others they provoke unwanted side effects such as inflammation and the elimination of antigen-bearing cells. Accordingly, the present invention further relates to TIM-1-binding proteins, including antibodies, with altered, e.g., reduced, effector functions.

Effector function of an anti-TIM-1 antibody of the present invention may be determined using one of many known assays. The anti-TIM-1 antibody's effector function may be reduced relative to a second anti-TIM-1 antibody. In some embodiments, the second anti-TIM-1 antibody may be any antibody that binds TIM-1 specifically. In other embodiments, the second TIM-1-specific antibody may be any of the antibodies of the invention, such as ARD5. In other embodiments, where the anti-TIM-1 antibody of interest has been modified to reduce effector function, the second anti-TIM-1 antibody may be the unmodified or parental version of the antibody.

Exemplary effector functions include Fc receptor binding, phagocytosis, apoptosis, pro-inflammatory responses, down-regulation of cell surface receptors (e.g. B cell receptor; BCR), etc. Other effector functions include antibody-dependent cell-mediated cytotoxicity (ADCC), whereby antibodies bind Fc receptors on cytotoxic T cells, natural killer (NK) cells, or macrophages leading to cell death, and complement-dependent cytotoxicity (CDC), which is cell death induced via activation of the complement cascade (reviewed in Daeron, Annu. Rev. Immunol. 15:203-234 (1997); Ward and Ghetie, Therapeutic Immunol. 2:77-94 (1995); and Ravetch and Kinet, Annu. Rev. Immunol. 9:457-492 (1991)). Such effector functions generally require the Fc region to be combined with a binding domain (e.g. an antibody variable domain) and can be assessed using standard assays that are known in the art (see, e.g., WO 05/018572, WO 05/003175, and U.S. Pat. No. 6,242,195). Effector functions can be avoided by using antibody fragments lacking the Fc domain such as Fab, Fab′2, or single chain Fv. An alternative has been to use the IgG4 subtype antibody, which binds to FcγRI but which binds poorly to C1q and FcγRII and RIII. The IgG2 subtype also has reduced binding to Fc receptors, but retains significant binding to the H131 allotype of FcγRIIa and to C1q. Thus, additional changes in the Fc sequence are required to eliminate binding to all the Fc receptors and to C1q.

Several antibody effector functions, including ADCC, are mediated by Fc receptors (FcRs), which bind the Fc region of an antibody. The affinity of an antibody for a particular FcR, and hence the effector activity mediated by the antibody, may be modulated by altering the amino acid sequence and/or post-translational modifications of the Fc and/or constant region of the antibody.

FcRs are defined by their specificity for immunoglobulin isotypes; Fc receptors for IgG antibodies are referred to as FcγR, for IgE as FcεR, for IgA as FcαR and so on. Three subclasses of FcγR have been identified: FcγRI (CD64), FcγRII (CD32) and FcγRIII (CD16). Both FcγRII and FcγRIII have two types: FcγRIIA (CD32) and FcγRIIB (CD32); and FcγRIIIA (CD16a) and FcγRIIIB (CD16b). Because each FcγR subclass is encoded by two or three genes, and alternative RNA splicing leads to multiple transcripts, a broad diversity in FcγR isoforms exists. For example, FcγRII (CD32) includes the isoforms 11a, 11b1, 11b2 11b3, and 11c.

The binding site on human and murine antibodies for FcγR has been previously mapped to the so-called “lower hinge region” consisting of residues 233-239 (EU index numbering as in Kabat et al., Sequences of Proteins of Immunological Interest, 5th Ed. Public Health Service, National Institutes of Health, Bethesda, Md. (1991), Woof et al. Molec. Immunol. 23:319-330 (1986); Duncan et al. Nature 332:563 (1988); Canfield and Morrison, J. Exp. Med. 173:1483-1491 (1991); Chappel et al., Proc. Natl. Acad. Sci. USA 88:9036-9040 (1991)). Of residues 233-239, P238 and 5239 are among those cited as possibly being involved in binding. Other previously cited areas possibly involved in binding to FcγR are: G316-K338 (human IgG) for human FcγRI (by sequence comparison only; no substitution mutants were evaluated) (Woof et al. Molec Immunol. 23:319-330 (1986)); K274-R301 (human IgG1) for human FcγRIII (based on peptides) (Sarmay et al. Molec. Immunol. 21:43-51 (1984)); and Y407-R416 (human IgG) for human FcγRIII (based on peptides) (Gergely et al. Biochem. Soc. Trans. 12:739-743 (1984) and Shields et al. J Biol Chem 276: 6591-6604 (2001), Lazar G A et al. Proc Natl Acad Sci 103: 4005-4010 (2006). These and other stretches or regions of amino acid residues involved in FcR binding may be evident to the skilled artisan from an examination of the crystal structures of Ig-FcR complexes (see, e.g., Sondermann et al. 2000 Nature 406(6793):267-73 and Sondermann et al. 2002 Biochem Soc Trans. 30(4):481-6). Accordingly, the anti-TIM-1 antibodies of the present invention include modifications of one or more of the aforementioned residues.

Other known approaches for reducing monoclonal antibody effector function include mutating amino acids on the surface of the monoclonal antibody that are involved in effector binding interactions (Lund, J., et al. (1991) J. Immunol. 147(8): 2657-62; Shields, R. L. et al. (2001) J. Biol. Chem. 276(9): 6591-604; and using combinations of different subtype sequence segments (e.g., IgG2 and IgG4 combinations) to give a greater reduction in binding to Fcγ receptors than either subtype alone (Armour et al., Eur. J. Immunol. (1999) 29: 2613-1624; Mol. Immunol. 40 (2003) 585-593). For example, sites of N-linked glycosylation can be removed as a means of reducing effector function.

A large number of Fc variants having altered and/or reduced affinities for some or all Fc receptor subtypes (and thus for effector functions) are known in the art. See, e.g., US 2007/0224188; US 2007/0148171; US 2007/0048300; US 2007/0041966; US 2007/0009523; US 2007/0036799; US 2006/0275283; US 2006/0235208; US 2006/0193856; US 2006/0160996; US 2006/0134105; US 2006/0024298; US 2005/0244403; US 2005/0233382; US 2005/0215768; US 2005/0118174; US 2005/0054832; US 2004/0228856; US 2004/132101; US 2003/158389; see also U.S. Pat. Nos. 7,183,387; 6,737,056; 6,538,124; 6,528,624; 6,194,551; 5,624,821; 5,648,260.

In CDC, the antibody-antigen complex binds complement, resulting in the activation of the complement cascade and generation of the membrane attack complex. Activation of the classical complement pathway is initiated by the binding of the first component of the complement system (C1q) to antibodies (of the appropriate subclass) which are bound to their cognate antigen; thus the activation of the complement cascade is regulated in part by the binding affinity of the immunoglobulin to C1q protein. To activate the complement cascade, it is necessary for C1q to bind to at least two molecules of IgG1, IgG2, or IgG3, but only one molecule of IgM, attached to the antigenic target (Ward and Ghetie, Therapeutic Immunology 2:77-94 (1995) p. 80). To assess complement activation, a CDC assay, e.g. as described in Gazzano-Santoro et al., J. Immunol. Methods 202:163 (1996), may be performed.

It has been proposed that various residues of the IgG molecule are involved in binding to C1q including the Glu318, Lys320 and Lys322 residues on the CH2 domain, amino acid residue 331 located on a turn in close proximity to the same beta strand, the Lys235 and Gly237 residues located in the lower hinge region, and residues 231 to 238 located in the N-terminal region of the CH2 domain (see e.g., Xu et al., J. Immunol. 150:152A (Abstract) (1993), WO94/29351; Tao et al, J. Exp. Med., 178:661-667 (1993); Brekke et al., Eur. J. Immunol., 24:2542-47 (1994); Burton et al; Nature, 288:338-344 (1980); Duncan and Winter, Nature 332:738-40 (1988); Idusogie et al J Immunol 164: 4178-4184 (2000; U.S. Pat. No. 5,648,260, and U.S. Pat. No. 5,624,821). As an example in IgG1, two mutations in the COOH terminal region of the CH2 domain of human IgG1—K322A and P329A—do not activate the CDC pathway and were shown to result in more than a 100 fold decrease in C1q binding (U.S. Pat. No. 6,242,195).

Thus, in certain embodiments of the invention, one or more of these residues may be modified, substituted, or removed or one or more amino acid residues may be inserted so as to decrease CDC activity of the TIM-1 antibodies provided herein. For example in some embodiments, it may be desirable to reduce or eliminate effector function(s) of the subject antibodies in order to reduce or eliminate the potential of further activating immune responses. Antibodies with decreased effector function may also reduce the risk of thromboembolic events in subjects receiving the antibodies.

In certain other embodiments, the present invention provides an anti-TIM-1 antibody that exhibits reduced binding to one or more FcR receptors but that maintains its ability to bind complement (e.g., to a similar or, in some embodiments, to a lesser extent than a native, non-variant, or parent anti-TIM-1 antibody). Accordingly, an anti-TIM-1 antibody of the present invention may bind and activate complement while exhibiting reduced binding to an FcR, such as, for example, FcγRIIa (e.g., FcγRIIa expressed on platelets). Such an antibody with reduced or no binding to FcγRIIa (such as FcγRIIa expressed on platelets, for example) but that can bind C1q and activate the complement cascade to at least some degree will reduce the risk of thromboembolic events while maintaining perhaps desirable effector functions. In alternative embodiments, an anti-TIM-1 antibody of the present invention exhibits reduced binding to one or more FcRs but maintains its ability to bind one or more other FcRs. See, for example, US 2007-0009523, 2006-0194290, 2005-0233382, 2004-0228856, and 2004-0191244, which describe various amino acid modifications that generate antibodies with reduced binding to FcRI, FcRII, and/or FcRIII, as well as amino acid substitutions that result in increased binding to one FcR but decreased binding to another FcR.

Accordingly, effector functions involving the constant region of an anti-TIM-1 antibody may be modulated by altering properties of the constant region, and the Fc region in particular. In certain embodiments, the anti-TIM-1 antibody having reduced effector function is compared with a second antibody with effector function and which may be a non-variant, native, or parent antibody comprising a native constant or Fc region that mediates effector function. In particular embodiments, effector function modulation includes situations in which an activity is abolished or completely absent.

A native sequence Fc or constant region comprises an amino acid sequence identical to the amino acid sequence of a Fc or constant chain region found in nature. Preferably, a control molecule used to assess relative effector function comprises the same type/subtype Fc region as does the test or variant antibody. A variant or altered Fc or constant region comprises an amino acid sequence which differs from that of a native sequence heavy chain region by virtue of at least one amino acid modification (such as, for example, post-translational modification, amino acid substitution, insertion, or deletion). Accordingly, the variant constant region may contain one or more amino acid substitutions, deletions, or insertions that results in altered post-translational modifications, including, for example, an altered glycosylation pattern. A parent antibody or Fc region is, for example, a variant having normal effector function used to construct a constant region (i.e., Fc) having altered, e.g., reduced, effector function.

Antibodies with altered (e.g., reduced or eliminated) effector function(s) may be generated by engineering or producing antibodies with variant constant, Fc, or heavy chain regions. Recombinant DNA technology and/or cell culture and expression conditions may be used to produce antibodies with altered function and/or activity. For example, recombinant DNA technology may be used to engineer one or more amino acid substitutions, deletions, or insertions in regions (such as, for example, Fc or constant regions) that affect antibody function including effector functions. Alternatively, changes in post-translational modifications, such as, e.g. glycosylation patterns (see below), may be achieved by manipulating the host cell and cell culture and expression conditions by which the antibody is produced.

Amino acid alterations, such as amino acid substitutions, can alter the effector function of the anti-TIM-1 antibodies of the present invention without affecting antigen binding affinity. The amino acid substitutions described above (e.g., Glu318, Kys320, Lys332, Lys235, Gly237, K332, and P329), for example, may be used to generate antibodies with reduced effector function.

In other embodiments, amino acid substitutions may be made for one or more of the following amino acid residues: 234, 235, 236, 237, 297, 318, 320, and 322 of the heavy chain constant region (see U.S. Pat. No. 5,624,821 and U.S. Pat. No. 5,648,260). Such substitutions may alter effector function while retaining antigen binding activity. An alteration at one or more of amino acids 234, 235, 236, and 237 can decrease the binding affinity of the Fc region for FcγRI receptor as compared to an unmodified or non-variant antibody Amino acid residues 234, 236, and/or 237 may be substituted with alanine, for example, and amino acid residue 235 may be substituted with glutamine, for example. In another embodiment, an anti-TIM-1 IgG1 antibody may comprise a substitution of Leu at position 234 with Ala, a substitution of Leu at position 235 with Glu, and a substitution of Gly at position 237 with Ala.

Additionally or alternatively, the Fc amino acid residues at 318, 320, and 322 may be altered. These amino acid residues, which are highly conserved in mouse and human IgGs, mediate complement binding. It has been shown that alteration of these amino acid residues reduces C1q binding but does not alter antigen binding, protein A binding, or the ability of the Fc to bind to mouse macrophages.

In another embodiment, an anti-TIM-1 antibody of the present invention is an IgG4 immunoglobulin comprising substitutions that reduce or eliminate effector function. The IgG4 Fc portion of an anti-TIM-1 antibody of the invention may comprise one or more of the following substitutions: substitution of proline for glutamate at residue 233, alanine or valine for phenylalanine at residue 234 and alanine or glutamate for leucine at residue 235 (EU numbering, Kabat, E. A. et al. (1991), supra). Further, removing the N-linked glycosylation site in the IgG4 Fc region by substituting Ala for Asn at residue 297 (EU numbering) may further reduce effector function and eliminate any residual effector activity that may exist. Another exemplary IgG4 mutant with reduced effector function is the IgG4 subtype variant containing the mutations S228P and L235E (PE mutation) in the heavy chain constant region. This mutation results in reduced effector function. See U.S. Pat. No. 5,624,821 and U.S. Pat. No. 5,648,260. Another exemplary mutation in the IgG4 context that reduces effector function is S228P/T229A, as described herein.

Other exemplary amino acid sequence changes in the constant region include but are not limited to the Ala-Ala mutation described by Bluestone et al. (see WO 94/28027 and WO 98/47531; also see Xu et al. 2000 Cell Immunol 200; 16-26). Thus in certain embodiments, anti-TIM-1 antibodies with mutations within the constant region including the Ala-Ala mutation may be used to reduce or abolish effector function. According to these embodiments, the constant region of an anti-TIM-1 antibody comprises a mutation to an alanine at position 234 or a mutation to an alanine at position 235. Additionally, the constant region may contain a double mutation: a mutation to an alanine at position 234 and a second mutation to an alanine at position 235.

In one embodiment, an anti-TIM-1 antibody comprises an IgG4 framework, wherein the Ala-Ala mutation would describe a mutation(s) from phenylalanine to alanine at position 234 and/or a mutation from leucine to alanine at position 235. In another embodiment, the anti-TIM-1 antibody comprises an IgG1 framework, wherein the Ala-Ala mutation would describe a mutation(s) from leucine to alanine at position 234 and/or a mutation from leucine to alanine at position 235. An anti-TIM-1 antibody may alternatively or additionally carry other mutations, including the point mutation K322A in the CH2 domain (Hezareh et al. 2001 J. Virol. 75: 12161-8).

Other exemplary amino acid substitutions are provided in WO 94/29351 (which is incorporated herein by reference in its entirety), which recites antibodies having mutations in the N-terminal region of the CH2 domain that alter the ability of the antibodies to bind to FcRI, thereby decreasing the ability of antibodies to bind to C1q which in turn decreases the ability of the antibodies to fix complement. Also see Cole et al. (J. Immunol. (1997) 159: 3613-3621), which describes mutations in the upper CH2 regions in IgG2 that result in lower FcR binding.

Methods of generating any of the aforementioned antibody variants comprising amino acid substitutions are well known in the art. These methods include, but are not limited to, preparation by site-directed (or oligonucleotide-mediated) mutagenesis, PCR mutagenesis, and cassette mutagenesis of a prepared DNA molecule encoding the antibody or at least the constant region of the antibody.

Site-directed mutagenesis is well known in the art (see, e.g., Carter et al. Nucleic Acids Res. 13:4431-4443 (1985) and Kunkel et al., Proc. Natl. Acad. Sci. USA 82:488 (1987)).

PCR mutagenesis is also suitable for making amino acid sequence variants of the starting polypeptide. See Higuchi, in PCR Protocols, pp. 177-183 (Academic Press, 1990); and Vallette et al., Nuc. Acids Res. 17:723-733 (1989). Another method for preparing sequence variants, cassette mutagenesis, is based on the technique described by Wells et al., Gene 34:315-323 (1985).

Another embodiment of the present invention relates to an anti-TIM-1 antibody with reduced effector function in which the antibody's Fc region, or portions thereof, is swapped with an Fc region (or with portions thereof) having naturally reduced effector inducing activity. For example, human IgG4 constant region exhibits reduced or no complement activation. Further, the different IgG molecules differ in their binding affinity for FcR, which may be due at least in part to the varying length and flexibility of the IgGs' hinge regions (which decreases in the order IgG3>IgG1>IgG4>IgG2). For example, IgG4 exhibits reduced or no binding to FcγRIIa. For examples of chimeric molecules and chimeric constant regions, see, e.g., Gillies et al. (Cancer Res. 1999, 59: 2159-2166) and Mueller et al. (Mol. Immunol. 1997, 34: 441-452).

The invention also relates to anti-TIM-1 antibodies with reduced effector function in which the Fc region is completely absent. Such antibodies may also be referred to as antibody derivatives and antigen-binding fragments of the present invention. Such derivatives and fragments may be fused to non-antibody protein sequences or non-protein structures, especially structures designed to facilitate delivery and/or bioavailability when administered to an animal, e.g., a human subject (see below).

As discussed above, changes within the hinge region also affect effector functions. For example, deletion of the hinge region may reduce affinity for Fc receptors and may reduce complement activation (Klein et al. 1981 PNAS USA 78: 524-528). The present disclosure therefore also relates to antibodies with alterations in the hinge region.

In particular embodiments, antibodies of the present invention may be modified to inhibit complement dependent cytotoxicity (CDC). Modulated CDC activity may be achieved by introducing one or more amino acid substitutions, insertions, or deletions in an Fc region of the antibody (see, e.g., U.S. Pat. No. 6,194,551 and U.S. Pat. No. 6,242,195). Alternatively or additionally, cysteine residue(s) may be introduced in the Fc region, thereby allowing interchain disulfide bond formation in this region. The homodimeric antibody thus generated may have improved or reduced internalization capability and/or increased or decreased complement-mediated cell killing. See Caron et al., J. Exp Med. 176:1191-1195 (1992) and Shopes, B. J. Immunol. 148:2918-2922 (1992), WO 99/51642, Duncan & Winter Nature 322: 738-40 (1988); U.S. Pat. No. 5,648,260; U.S. Pat. No. 5,624,821; and WO 94/29351.

It is further understood that effector function may vary according to the binding affinity of the antibody. For example, antibodies with high affinity may be more efficient in activating the complement system compared to antibodies with relatively lower affinity (Marzocchi-Machado et al. 1999 Immunol Invest 28: 89-101). Accordingly, an antibody may be altered such that the binding affinity for its antigen is reduced (e.g., by changing the variable regions of the antibody by methods such as substitution, addition, or deletion of one or more amino acid residues). An antibody with reduced binding affinity may exhibit reduced effector functions, including, for example, reduced ADCC and/or CDC.

Anti-TIM-1 antibodies of the present invention with reduced effector function include antibodies with reduced binding affinity for one or more Fc receptors (FcRs) relative to a parent or non-variant anti-TIM-1 antibody. Accordingly, anti-TIM-1 antibodies with reduced FcR binding affinity includes anti-TIM-1 antibodies that exhibit a 1.5-fold, 2-fold, 2.5-fold, 3-fold, 4-fold, or 5-fold or higher decrease in binding affinity to one or more Fc receptors compared to a parent or non-variant anti-TIM-1 antibody. In some embodiments, an anti-TIM-1 antibody with reduced effector function binds to an FcR with about 10-fold less affinity relative to a parent or non-variant antibody. In other embodiments, an anti-TIM-1 antibody with reduced effector function binds to an FcR with about 15-fold less affinity or with about 20-fold less affinity relative to a parent or non-variant antibody. The FcR receptor may be one or more of FcγRI (CD64), FcγRII (CD32), and FcγRIII, and isoforms thereof, and FcεR, FcμR, FcδR, and/or an FcαR. In particular embodiments, an anti-TIM-1 antibody with reduced effector function exhibits a 1.5-fold, 2-fold, 2.5-fold, 3-fold, 4-fold, or 5-fold or higher decrease in binding affinity to FcγRIIa.

Accordingly, in certain embodiments, an anti-TIM-1 antibody of the present invention exhibits reduced binding to a complement protein relative to a second anti-TIM-1 antibody. In certain embodiments, an anti-TIM-1 antibody of the invention exhibits reduced binding by a factor of about 1.5-fold or more, about 2-fold or more, about 3-fold or more, about 4-fold or more, about 5-fold or more, about 6-fold or more, about 7-fold or more, about 8-fold or more, about 9-fold or more, about 10-fold or more, or about 15-fold or more, relative to a second anti-TIM-1 antibody.

Certain embodiments of the present invention relate to an anti-TIM-1 antibody comprising one or more heavy chain CDR sequences selected from CDR-H1 of SEQ ID NO:4, CDR-H2 of SEQ ID NO:4 and CDR-H3 of SEQ ID NO:4, wherein the antibody further comprises a variant Fc region that confers reduced effector function compared to a native or parental Fc region. In further embodiments, the anti-TIM-1 antibody comprises at least two of the CDRs, and in other embodiments the antibody comprises all three of the heavy chain CDR sequences.

Other embodiments of the present invention relate to an anti-TIM-1 antibody comprising one or more light chain CDR sequences selected from CDR-L1 of SEQ ID NO:6, CDR-L2 of SEQ ID NO:6 and CDR-L3 of SEQ ID NO:6, the antibody further comprising a variant Fc region that confers reduced effector function compared to a native or parental Fc region. In further embodiments, the anti-TIM-1 antibody comprises at least two of the light chain CDRs, and in other embodiments the antibody comprises all three of the light chain CDR sequences.

In further embodiments of the present invention, the anti-TIM-1 antibody with reduced effector function comprises all three light chain CDR sequences of SEQ ID NO:6 and comprises all three heavy chain CDR sequences of SEQ ID NO:4.

In other embodiments, the invention relates to an anti-TIM-1 antibody comprising a V_{L} sequence comprising SEQ ID NO:6, the antibody further comprising a variant Fc region that confers reduced effector function compared to a native or parental Fc region.

In other embodiments, the invention relates to an anti-TIM-1 antibody comprising a V_{H} sequence comprising SEQ ID NO:4, the antibody further comprising a variant Fc region that confers reduced effector function compared to a native or parental Fc region.

Anti-TIM-1 Antibodies with Altered Glycosylation

Glycan removal produces a structural change that should greatly reduce binding to all members of the Fc receptor family across species. In glycosylated antibodies, including anti-TIM-1 antibodies, the glycans (oligosaccharides) attached to the conserved N-linked site in the CH2 domains of the Fc dimer are enclosed between the CH2 domains, with the sugar residues making contact with specific amino acid residues on the opposing CH2 domain. Different glycosylation patterns are associated with different biological properties of antibodies (Jefferis and Lund, 1997, Chem. Immunol., 65: 111-128; Wright and Morrison, 1997, Trends Biotechnol., 15: 26-32). Certain specific glycoforms confer potentially advantageous biological properties. Loss of the glycans changes spacing between the domains and increases their mobility relative to each other and is expected to have an inhibitory effect on the binding of all members of the Fc receptor family. For example, in vitro studies with various glycosylated antibodies have demonstrated that removal of the CH2 glycans alters the Fc structure such that antibody binding to Fc receptors and the complement protein C1Q are greatly reduced. Another known approach to reducing effector functions is to inhibit production of or remove the N-linked glycans at position 297 (EU numbering) in the CH2 domain of the Fc (Nose et al., 1983 PNAS 80: 6632; Leatherbarrow et al., 1985 Mol. Immunol. 22: 407; Tao et al., 1989 J. Immunol. 143: 2595; Lund et al., 1990 Mol. Immunol. 27: 1145; Dorai et al., 1991 Hybridoma 10:211; Hand et al., 1992 Cancer Immunol. Immunother. 35:165; Leader et al., 1991 Immunology 72: 481; Pound et al., 1993 Mol. Immunol. 30:233; Boyd et al., 1995 Mol. Immunol. 32: 1311). It is also known that different glycoforms can profoundly affect the properties of a therapeutic, including pharmacokinetics, pharmacodynamics, receptor-interaction and tissue-specific targeting (Graddis et al., 2002, Curr Pharm Biotechnol. 3: 285-297). In particular, for antibodies, the oligosaccharide structure can affect properties relevant to protease resistance, the serum half-life of the antibody mediated by the FcRn receptor, phagocytosis and antibody feedback, in addition to effector functions of the antibody (e.g., binding to the complement complex C1, which induces CDC, and binding to FcγR receptors, which are responsible for modulating the ADCC pathway) (Nose and Wigzell, 1983; Leatherbarrow and Dwek, 1983; Leatherbarrow et al., 1985; Walker et al., 1989; Carter et al., 1992, PNAS, 89: 4285-4289).

Accordingly, another means of modulating effector function of antibodies includes altering glycosylation of the antibody constant region. Altered glycosylation includes, for example, a decrease or increase in the number of glycosylated residues, a change in the pattern or location of glycosylated residues, as well as a change in sugar structure(s). The oligosaccharides found on human IgGs affects their degree of effector function (Raju, T. S. BioProcess International April 2003. 44-53); the microheterogeneity of human IgG oligosaccharides can affect biological functions such as CDC and ADCC, binding to various Fc receptors, and binding to C1q protein (Wright A. & Morrison S L. TIBTECH 1997, 15 26-32; Shields et al. J Biol. Chem. 2001 276(9):6591-604; Shields et al. J Biol. Chem. 2002; 277(30):26733-40; Shinkawa et al. J Biol. Chem. 2003 278(5):3466-73; Umana et al. Nat. Biotechnol. 1999 February; 17(2): 176-80). For example, the ability of IgG to bind C1q and activate the complement cascade may depend on the presence, absence or modification of the carbohydrate moiety positioned between the two CH2 domains (which is normally anchored at Asn297) (Ward and Ghetie, Therapeutic Immunology 2:77-94 (1995).

Glycosylation sites in an Fc-containing polypeptide, for example an antibody such as an IgG antibody, may be identified by standard techniques. The identification of the glycosylation site can be experimental or based on sequence analysis or modeling data. Consensus motifs, that is, the amino acid sequence recognized by various glycosyl transferases, have been described. For example, the consensus motif for an N-linked glycosylation motif is frequently NXT or NXS, where X can be any amino acid except proline. Several algorithms for locating a potential glycosylation motif have also been described. Accordingly, to identify potential glycosylation sites within an antibody or Fc-containing fragment, the sequence of the antibody is examined, for example, by using publicly available databases such as the website provided by the Center for Biological Sequence Analysis (see NetNGlyc services for predicting N-linked glycosylation sites and NetOGlyc services for predicting O-linked glycosylation sites).

In vivo studies have confirmed the reduction in the effector function of aglycosyl antibodies. For example, an aglycosyl anti-CD8 antibody is incapable of depleting CD8-bearing cells in mice (Isaacs, 1992 J. Immunol. 148: 3062) and an aglycosyl anti-CD3 antibody does not induce cytokine release syndrome in mice or humans (Boyd, 1995 supra; Friend, 1999 Transplantation 68:1632).

Importantly, while removal of the glycans in the CH2 domain appears to have a significant effect on effector function, other functional and physical properties of the antibody remain unaltered. Specifically, it has been shown that removal of the glycans had little to no effect on serum half-life and binding to antigen (Nose, 1983 supra; Tao, 1989 supra; Dorai, 1991 supra; Hand, 1992 supra; Hobbs, 1992 Mol. Immunol. 29:949).

Although there is in vivo validation of the aglycosyl approach, there are reports of residual effector function with aglycosyl monoclonal antibodies (see, e.g., Pound, J. D. et al. (1993) Mol. Immunol. 30(3): 233-41; Dorai, H. et al. (1991) Hybridoma 10(2): 211-7). Armour et al. show residual binding to FcγRIIa and FcγRIIb proteins (Eur. J. Immunol. (1999) 29: 2613-1624; Mol. Immunol. 40 (2003) 585-593). Thus a further decrease in effector function, particularly complement activation, may be important to guarantee complete ablation of activity in some instances. For that reason, aglycosyl forms of IgG2 and IgG4 and a G1/G4 hybrid are envisioned as being useful in methods and antibody compositions of the invention having reduced effector functions.

The anti-TIM-1 antibodies of the present invention may be modified or altered to elicit reduced effector function(s) (compared to a second TIM-1-specific antibody) while optionally retaining the other valuable attributes of the Fc portion.

Accordingly, in certain embodiments, the present invention relates to aglycosyl anti-TIM-1 antibodies with decreased effector function, which are characterized by a modification at the conserved N-linked site in the CH2 domains of the Fc portion of the antibody. A modification of the conserved N-linked site in the CH2 domains of the Fc dimer can lead to aglycosyl anti-TIM-1 antibodies. Examples of such modifications include mutation of the conserved N-linked site in the CH2 domains of the Fc dimer, removal of glycans attached to the N-linked site in the CH2 domains, and prevention of glycosylation. For example, an aglycosyl anti-TIM-1 antibody may be created by changing the canonical N-linked Asn site in the heavy chain CH2 domain to a Gln residue (see, for example, WO 05/03175 and US 2006-0193856).

In one embodiment of present invention, the modification comprises a mutation at the heavy chain glycosylation site to prevent glycosylation at the site. Thus, in one embodiment of this invention, the aglycosyl anti-TIM-1 antibodies are prepared by mutation of the heavy chain glycosylation site, i.e., mutation of N298Q (N297 using Kabat EU numbering) and expressed in an appropriate host cell. For example, this mutation may be accomplished by following the manufacturer's recommended protocol for unique site mutagenesis kit from Amersham-Pharmacia Biotech® (Piscataway, N.J., USA).

The mutated antibody can be stably expressed in a host cell (e.g. NSO or CHO cell) and then purified. As one example, purification can be carried out using Protein A and gel filtration chromatography. It will be apparent to those of skill in the art that additional methods of expression and purification may also be used.

In another embodiment of the present invention, the aglycosyl anti-TIM-1 antibodies have decreased effector function, wherein the modification at the conserved N-linked site in the CH2 domains of the Fc portion of said antibody or antibody derivative comprises the removal of the CH2 domain glycans, i.e., deglycosylation. These aglycosyl anti-TIM-1 antibodies may be generated by conventional methods and then deglycosylated enzymatically. Methods for enzymatic deglycosylation of antibodies are well known to those of skill in the art (Williams, 1973; Winkelhake & Nicolson, 1976 J. Biol. Chem. 251:1074-80.).

In another embodiment of this invention, deglycosylation may be achieved by growing host cells which produce the antibodies in culture medium comprising a glycosylation inhibitor such as tunicamycin (Nose & Wigzell, 1983). That is, the modification is the reduction or prevention of glycosylation at the conserved N-linked site in the CH2 domains of the Fc portion of said antibody.

In other embodiments of this invention, recombinant X polypeptides (or cells or cell membranes containing such polypeptides) may be used as an antigen to generate an anti-TIM-1 antibody or antibody derivatives, which may then be deglycosylated.

In alternative embodiments, agyclosyl anti-TIM-1 antibodies or anti-TIM-1 antibodies with reduced glycosylation of the present invention, may be produced by the method described in Taylor et al. (WO 05/18572 and US 2007-0048300). For example, in one embodiment, an anti-TIM-1 aglycosyl antibody may be produced by altering a first amino acid residue (e.g., by substitution, insertion, deletion, or by chemical modification), wherein the altered first amino acid residue inhibits the glycosylation of a second residue by either steric hindrance or charge or both. In certain embodiments, the first amino acid residue is modified by amino acid substitution. In further embodiments, the amino acid substitution is selected from the group consisting of Gly, Ala, Val, Leu, Ile, Phe, Asn, Gln, Trp, Pro, Ser, Thr, Tyr, Cys, Met, Asp, Glu, Lys, Arg, and His. In other embodiments, the amino acid substitution is a non-traditional amino acid residue. The second amino acid residue may be near or within a glycosylation motif, for example, an N-linked glycosylation motif that contains the amino acid sequence NXT or NXS. In one exemplary embodiment, the first amino acid residue is amino acid 299 and the second amino acid residue is amino acid 297, according to the Kabat numbering. For example, the first amino acid substitution may be T299A, T299N, T299G, T299Y, T299C, T299H, T299E, T299D, T299K, T299R, T299G, T299I, T299L, T299M, T299F, T299P, T299W, and T299V, according to the Kabat numbering. In particular embodiments, the amino acid substitution is T299C.

Effector function may also be reduced by modifying an antibody of the present invention such that the antibody contains a blocking moiety. Exemplary blocking moieties include moieties of sufficient steric bulk and/or charge such that reduced glycosylation occurs, for example, by blocking the ability of a glycosidase to glycosylate the polypeptide. The blocking moiety may additionally or alternatively reduce effector function, for example, by inhibiting the ability of the Fc region to bind a receptor or complement protein. In some embodiments, the present invention relates to a TIM-1-binding protein, e.g., an anti-TIM-1 antibody, comprising a variant Fc region, the variant Fc region comprising a first amino acid residue and an N-glycosylation site, the first amino acid residue modified with side chain chemistry to achieve increased steric bulk or increased electrostatic charge compared to the unmodified first amino acid residue, thereby reducing the level of or otherwise altering glycosylation at the N-glycosylation site. In certain of these embodiments, the variant Fc region confers reduced effector function compared to a control, non-variant Fc region. In further embodiments, the side chain with increased steric bulk is a side chain of an amino acid residue selected from the group consisting of Phe, Trp, H is, Glu, Gln, Arg, Lys, Met and Tyr. In yet further embodiments, the side chain chemistry with increased electrostatic charge is a side chain of an amino acid residue selected from the group consisting of Asp, Glu, Lys, Arg, and His.

Accordingly, in one embodiment, glycosylation and Fc binding can be modulated by substituting T299 with a charged side chain chemistry such as D, E, K, or R. The resulting antibody will have reduced glycosylation as well as reduced Fc binding affinity to an Fc receptor due to unfavorable electrostatic interactions.

In another embodiment, a T299C variant antibody, which is both aglycosylated and capable of forming a cysteine adduct, may exhibit less effector function (e.g., FcγRI binding) compared to its aglycosylated antibody counterpart (see, e.g., WO 05/18572). Accordingly, alteration of a first amino acid proximal to a glycosylation motif can inhibit the glycosylation of the antibody at a second amino acid residue; when the first amino acid is a cysteine residue, the antibody may exhibit even further reduced effector function. In addition, inhibition of glycosylation of an antibody of the IgG4 subtype may have a more profound affect on FcγRI binding compared to the effects of agycosylation in the other subtypes.

In additional embodiments, the present invention relates to anti-TIM-1 antibodies with altered glycosylation that exhibit reduced binding to one or more FcR receptors and that optionally also exhibit increased or normal binding to one or more Fc receptors and/or complement—e.g., antibodies with altered glycosylation that at least maintain the same or similar binding affinity to one or more Fc receptors and/or complement as a native, control anti-TIM-1 antibody). For example, anti-TIM-1 antibodies with predominantly Man₅GlcNAc₂N-glycan as the glycan structure present (e.g., wherein Man₅GlcNAc₂N-glycan structure is present at a level that is at least about 5 mole percent more than the next predominant glycan structure of the Ig composition) may exhibit altered effector function compared to an anti-TIM-1 antibody population wherein Man₅GlcNAc₂N-glycan structure is not predominant Antibodies with predominantly this glycan structure exhibit decreased binding to FcγRIIa and FcγRIIb, increased binding to FcγRIIIa and FcγRIIIb, and increased binding to C1q subunit of the C1 complex (see US 2006-0257399). This glycan structure, when it is the predominant glycan structure, confers increased ADCC, increased CDC, increased serum half-life, increased antibody production of B cells, and decreased phagocytosis by macrophages.

In general, the glycosylation structures on a glycoprotein will vary depending upon the expression host and culturing conditions (Raju, T S. BioProcess International April 2003. 44-53). Such differences can lead to changes in both effector function and pharmacokinetics (Israel et al. Immunology. 1996; 89(4):573-578; Newkirk et al. P. Clin. Exp. 1996; 106(2):259-64). For example, galactosylation can vary with cell culture conditions, which may render some immunoglobulin compositions immunogenic depending on their specific galactose pattern (Patel et al., 1992. Biochem J. 285: 839-845). The oligosaccharide structures of glycoproteins produced by non-human mammalian cells tend to be more closely related to those of human glycoproteins. Further, protein expression host systems may be engineered or selected to express a predominant Ig glycoform or alternatively may naturally produce glycoproteins having predominant glycan structures. Examples of engineered protein expression host systems producing a glycoprotein having a predominant glycoform include gene knockouts/mutations (Shields et al., 2002, JBC, 277: 26733-26740); genetic engineering in (Umana et al., 1999, Nature Biotech., 17: 176-180) or a combination of both. Alternatively, certain cells naturally express a predominant glycoform—for example, chickens, humans and cows (Raju et al., 2000, Glycobiology, 10: 477-486). Thus, the expression of an anti-TIM-1 antibody or antibody composition having altered glycosylation (e.g., predominantly one specific glycan structure) can be obtained by one skilled in the art by selecting at least one of many expression host systems. Protein expression host systems that may be used to produce anti-TIM-1 antibodies of the present invention include animal, plant, insect, bacterial cells and the like. For example, US 2007-0065909, 2007-0020725, and 2005-0170464 describe producing aglycosylated immunoglobulin molecules in bacterial cells. As a further example, Wright and Morrison produced antibodies in a CHO cell line deficient in glycosylation (1994 J Exp Med 180: 1087-1096) and showed that antibodies produced in this cell line were incapable of complement-mediated cytolysis. Other examples of expression host systems found in the art for production of glycoproteins include: CHO cells: Raju WO 99/22764 and Presta WO 03/35835; hybridoma cells: Trebak et al., 1999, J. Immunol. Methods, 230: 59-70; insect cells: Hsu et al., 1997, JBC, 272:9062-970, and plant cells: Gerngross et al., WO 04/74499. To the extent that a given cell or extract has resulted in the glycosylation of a given motif, art recognized techniques for determining if the motif has been glycosylated are available, for example, using gel electrophoresis and/or mass spectroscopy.

Additional methods for altering glycosylation sites of antibodies are described, e.g., in U.S. Pat. No. 6,350,861 and U.S. Pat. No. 5,714,350, WO 05/18572 and WO 05/03175; these methods can be used to produce anti-TIM-1 antibodies of the present invention with altered, reduced, or no glycosylation.

The aglycosyl anti-TIM-1 antibodies with reduced effector function may be antibodies that comprise modifications or that may be conjugated to comprise a functional moiety. Such moieties include a blocking moiety (e.g., a PEG moiety, cysteine adducts, etc.), a detectable moiety (e.g., fluorescent moieties, radioisotopic moieties, radiopaque moieties, etc., including diagnostic moieties), a therapeutic moiety (e.g., cytotoxic agents, anti-inflammatory agents, immunomodulatory agents, anti-infective agents, anti-cancer agents, anti-neurodegenerative agents, radionuclides, etc.), and/or a binding moiety or bait (e.g., that allows the antibody to be pre-targeted to a tumor and then to bind a second molecule, composed of the complementary binding moiety or prey and a detectable moiety or therapeutic moeity, as described above).

### TIM-1-Associated Disorders

An anti-TIM-1 antibody described herein can be used to treat or prevent a variety of immunological disorders, such as inflammatory and autoimmune disorders.

The term “treating” refers to administering a composition described herein in an amount, manner, and/or mode effective to improve a condition, symptom, or parameter associated with a disorder or to prevent progression or exacerbation of the disorder (including secondary damage caused by the disorder) to either a statistically significant degree or to a degree detectable to one skilled in the art.

A subject who is at risk for, diagnosed with, or who has one of these disorders can be administered an anti-TIM-1 antibody in an amount and for a time to provide an overall therapeutic effect. The anti-TIM-1 antibody can be administered alone (monotherapy) or in combination with other agents (combination therapy). In the case of a combination therapy, the amounts and times of administration can be those that provide, e.g., an additive or a synergistic therapeutic effect. Further, the administration of the anti-TIM-1 antibody (with or without the second agent) can be used as a primary, e.g., first line treatment, or as a secondary treatment, e.g., for subjects who have an inadequate response to a previously administered therapy (i.e., a therapy other than one with an anti-TIM-1 antibody). In some embodiments, the combination therapy includes the use of two or more anti-TIM-1 antibodies, e.g., at least one of the anti-TIM-1 antibodies described herein in combination with another anti-TIM-1 antibody, e.g., two or more of the anti-TIM-1 antibodies described herein.

Diseases or conditions treatable with an anti-TIM-1 antibody described herein include, e.g., ischemia-reperfusion injury (e.g., organ ischemia-reperfusion injury such as liver or renal ischemia-reperfusion injury), allergy, asthma, inflammatory bowel disease (IBD), Chron's disease, transplant rejection, pancreatitis, and delayed type hypersensitivity (DTH).

Additional diseases or conditions treatable with an anti-TIM-1 antibody described herein include, e.g., autoimmune disorders.

Systematic lupus erythromatosis (SLE; lupus) is a TH-2 mediated autoimmune disorder characterized by high levels of autoantibodies directed against intracellular antigens such as double stranded DNA, single stranded DNA, and histones.

Examples of other organ-specific or systemic autoimmune diseases suitable for treatment with an anti-TIM-1 antibody described herein include myasthenia gravis, autoimmune hemolytic anemia, Chagas' disease, Graves disease, idiopathic thrombocytopenia purpura (ITP), Wegener's Granulomatosis, poly-arteritis Nodosa and Rapidly Progressive Crescentic Glomerulonephritis. See, e.g., Benjamini et al., 1996, Immunology, A Short Course, Third Ed. (Wiley-Liss, New York). In addition, rheumatoid arthritis (RA) is suitable for treatment with an anti-TIM-1 antibody described herein.

Additional diseases or conditions treatable with an anti-TIM-1 antibody described herein include, e.g., Graft-Versus Host Disease (GVHD). GVHD exemplifies a T cell-mediated condition that can be treated using an anti-TIM-1 antibody described herein. GVHD is initiated when donor T cells recognize host antigens as foreign. GVHD, often a fatal consequence of bone marrow transplantation (BMT) in human patients, can be acute or chronic. Acute and chronic forms of GVHD exemplify the development of antigen specific Th1 and Th2 responses, respectively. Acute GVHD occurs within the first two months following BMT, and is characterized by donor cytotoxic T cell-mediated damage to skin, gut, liver, and other organs. Chronic GVHD appears later (over 100 days post-BMT) and is characterized by hyperproduction of immunoglobulin (Ig), including autoantibodies, and damage to the skin, kidney, and other organs caused by Ig-deposition. Nearly 90% of acute GVHD patients go on to develop chronic GVHD. Chronic GVHD appears to be a Th2 T cell mediated disease (De Wit et al., 1993, J. Immunol. 150:361-366). Acute GVHD is a Th1 mediated disease (Krenger et al., 1996, Immunol. Res. 15:50-73; Williamson et al., 1996, J. Immunol. 157:689-699). T cell cytotoxicity is a characteristic of acute GVHD. The consequence of donor anti-host cytotoxicity can be seen in various ways. First, host lymphocytes are rapidly destroyed, such that mice experiencing acute GVHD are profoundly immunosuppressed. Second, donor lymphocytes become engrafted and expand in the host spleen, and their cytotoxic activity can be directly measured in vitro by taking advantage of cell lines that express the host antigens that can be recognized (as foreign) by the donor cells. Third, the disease becomes lethal as additional tissues and cell populations are destroyed.

Additional diseases or conditions treatable with an anti-TIM-1 antibody described herein include, e.g., atopic disorders. Atopic disorders are characterized by the expression by immune system cells, including acivated T cells and APC, of cytokines, chemokines, and other molecules which are characteristic of Th2 responses, such as the IL-4, IL-5 and IL-13 cytokines, among others. Such atopic disorders therefore will be amenable to treatment with an anti-TIM-1 antibody described herein. Atopic disorders include airway hypersensitivity and distress syndromes, atopic dermatitis, contact dermatitis, urticaria, allergic rhinitis, angioedema, latex allergy, and an allergic lung disorder (e.g., asthma, allergic bronchopulmonary aspergillosis, and hypersensitivity pneumonitis).

Additional diseases or conditions treatable with an anti-TIM-1 antibody described herein include, e.g., numerous immune or inflammatory disorders Immune or inflammatory disorders include, but are not limited to, allergic rhinitis, autoimmune hemolytic anemia; acanthosis nigricans; Addison's disease; alopecia greata; alopecia universalis; amyloidosis; anaphylactoid purpura; anaphylactoid reaction; aplastic anemia; ankylosing spondylitis; arteritis, cranial; arteritis, giant cell; arteritis, Takayasu's; arteritis, temporal; ataxia-telangiectasia; autoimmune oophoritis; autoimmune orchitis; autoimmune polyendocrine failure; Behcet's disease; Berger's disease; Buerger's disease; bronchitis; bullous pemphigus; candidiasis, chronic mucocutaneous; Caplan's syndrome; post-myocardial infarction syndrome; post-pericardiotomy syndrome; carditis; celiac sprue; Chagas's disease; Chediak-Higashi syndrome; Churg-Strauss disease; Cogan's syndrome; cold agglutinin disease; CREST syndrome; Crohn's disease; cryoglobulinemia; cryptogenic fibrosing alveolitis; dermatitis herpetifomis; dermatomyositis; diabetes mellitus; Diamond-Blackfan syndrome; DiGeorge syndrome; discoid lupus erythematosus; eosinophilic fasciitis; episcleritis; drythema elevatum diutinum; erythema marginatum; erythema multiforme; erythema nodosum; Familial Mediterranean fever; Felty's syndrome; pulmonary fibrosis; glomerulonephritis, anaphylactoid; glomerulonephritis, autoimmune; glomerulonephritis, post-streptococcal; glomerulonephritis, post-transplantation; glomerulopathy, membranous; Goodpasture's syndrome; granulocytopenia, immune-mediated; granuloma annulare; granulomatosis, allergic; granulomatous myositis; Grave's disease; Hashimoto's thyroiditis; hemolytic disease of the newborn; hemochromatosis, idiopathic; Henoch-Schoenlein purpura; hepatitis, chronic active and chronic progressive; histiocytosis X; hypereosinophilic syndrome; idiopathic thrombocytopenic purpura; Job's syndrome; juvenile dermatomyositis; juvenile rheumatoid arthritis (Juvenile chronic arthritis); Kawasaki's disease; keratitis; keratoconjunctivitis sicca; Landry-Guillain-Barre-Strohl syndrome; leprosy, lepromatous; Loeffler's syndrome; lupus; Lyell's syndrome; lyme disease; lymphomatoid granulomatosis; mastocytosis, systemic; mixed connective tissue disease; mononeuritis multiplex; Muckle-Wells syndrome; mucocutaneous lymph node syndrome; mucocutaneous lymph node syndrome; multicentric reticulohistiocytosis; multiple sclerosis; myasthenia gravis; mycosis fungoides; necrotizing vasculitis, systemic; nephrotic syndrome; overlap syndrome; panniculitis; paroxysmal cold hemoglobinuria; paroxysmal nocturnal hemoglobinuria; pemphigoid; pemphigus; pemphigus erythematosus; pemphigus foliaceus; pemphigus vulgaris; pigeon breeder's disease; polyarteritis nodosa; polymyalgia rheumatic; polymyositis; polyneuritis, idiopathic; portuguese familial polyneuropathies; pre-eclampsia/eclampsia; primary biliary cirrhosis; progressive systemic sclerosis (scleroderma); psoriasis; psoriatic arthritis; pulmonary alveolar proteinosis; pulmonary fibrosis, Raynaud's phenomenon/syndrome; Reidel's thyroiditis; Reiter's syndrome, relapsing polychrondritis; rheumatic fever; rheumatoid arthritis; sarcoidosis; scleritis; sclerosing cholangitis; serum sickness; Sezary syndrome; Sjogren's syndrome; Stevens-Johnson syndrome; Still's disease; subacute sclerosing panencephalitis; sympathetic ophthalmia; systemic lupus erythematosus; yransplant rejection; ulcerative colitis; undifferentiated connective tissue disease; urticaria, chronic; urticaria, cold; uveitis; vitiligo; Weber-Christian disease; Wegener's granulomatosis, or Wiskott-Aldrich syndrome.

### Pharmaceutical Compositions

An anti-TIM-1 antibody (such as an antibody described herein) can be formulated as a pharmaceutical composition for administration to a subject, e.g., to treat a disorder described herein. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. The composition can include a pharmaceutically acceptable salt, e.g., an acid addition salt or a base addition salt (see e.g., Berge, S. M., et al. (1977) J. Pharm. Sci. 66:1-19).

Pharmaceutical formulation is a well-established art, and is further described, e.g., in Gennaro (ed.), Remington: The Science and Practice of Pharmacy, 20^{th} ed., Lippincott, Williams & Wilkins (2000) (ISBN: 0683306472); Ansel et al., Pharmaceutical Dosage Forms and Drug Delivery Systems, 7^{th} Ed., Lippincott Williams & Wilkins Publishers (1999) (ISBN: 0683305727); and Kibbe (ed.), Handbook of Pharmaceutical Excipients American Pharmaceutical Association, 3^{rd} ed. (2000) (ISBN: 091733096X).

The pharmaceutical compositions may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form can depend on the intended mode of administration and therapeutic application. Typically compositions for the agents described herein are in the form of injectable or infusible solutions.

In one embodiment, the anti-TIM-1 antibody is formulated with excipient materials, such as sodium chloride, sodium dibasic phosphate heptahydrate, sodium monobasic phosphate, and a stabilizer. It can be provided, for example, in a buffered solution at a suitable concentration and can be stored at 2-8° C.

Such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In certain embodiments, the anti-TIM-1 antibody may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York (1978).

An anti-TIM-1 antibody can be modified, e.g., with a moiety that improves its stabilization and/or retention in circulation, e.g., in blood, serum, or other tissues, e.g., by at least 1.5, 2, 5, 10, or 50 fold.

For example, the anti-TIM-1 antibody can be associated with (e.g., conjugated to) a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 Daltons (or about 1,000 to about 15,000, and 2,000 to about 12,500) can be used.

For example, the anti-TIM-1 antibody can be conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene; polymethacrylates; carbomers; and branched or unbranched polysaccharides.

### Administration

The anti-TIM-1 antibody can be administered to a subject, e.g., a subject in need thereof, for example, a human subject, by a variety of methods. For many applications, the route of administration is one of: intravenous injection or infusion (IV), subcutaneous injection (SC), intraperitoneally (IP), or intramuscular injection. It is also possible to use intra-articular delivery. Other modes of parenteral administration can also be used. Examples of such modes include: intraarterial, intrathecal, intracapsular, intraorbital, intracardiac, intradermal, transtracheal, subcuticular, intraarticular, subcapsular, subarachnoid, intraspinal, and epidural and intrasternal injection. In some cases, administration can be oral.

The route and/or mode of administration of the antibody can also be tailored for the individual case, e.g., by monitoring the subject, e.g., using tomographic imaging, e.g., to visualize a tumor.

The antibody can be administered as a fixed dose, or in a mg/kg dose. The dose can also be chosen to reduce or avoid production of antibodies against the anti-TIM-1 antibody. Dosage regimens are adjusted to provide the desired response, e.g., a therapeutic response or a combinatorial therapeutic effect. Generally, doses of the anti-TIM-1 antibody (and optionally a second agent) can be used in order to provide a subject with the agent in bioavailable quantities. For example, doses in the range of 0.1-100 mg/kg, 0.5-100 mg/kg, 1 mg/kg-100 mg/kg, 0.5-20 mg/kg, 0.1-10 mg/kg, or 1-10 mg/kg can be administered. Other doses can also be used.

A composition may comprise about 10 to 100 mg/ml or about 50 to 100 mg/ml or about 100 to 150 mg/ml or about 100 to 200 mg/ml of antibody.

In certain embodiments, the anti-TIM-1 antibody in a composition is predominantly in monomeric form, e.g., at least about 90%, 92%, 94%, 96%, 98%, 98.5% or 99% in monomeric form. Certain anti-TIM-1 antibody compositions may comprise less than about 5, 4, 3, 2, 1, 0.5, 0.3 or 0.1% aggregates, as detected, e.g., by UV at A280 nm. Certain anti-TIM-1 antibody compositions comprise less than about 5, 4, 3, 2, 1, 0.5, 0.3, 0.2 or 0.1% fragments, as detected, e.g., by UV at A280 nm.

Dosage unit form or “fixed dose” as used herein refers to physically discrete units suited as unitary dosages for the subjects to be treated; each unit contains a predetermined quantity of active compound calculated to produce the desired therapeutic effect in association with the required pharmaceutical carrier and optionally in association with the other agent. Single or multiple dosages may be given. Alternatively, or in addition, the antibody may be administered via continuous infusion.

An anti-TIM-1 antibody dose can be administered, e.g., at a periodic interval over a period of time (a course of treatment) sufficient to encompass at least 2 doses, 3 doses, 5 doses, 10 doses, or more, e.g., once or twice daily, or about one to four times per week, or preferably weekly, biweekly (every two weeks), every three weeks, monthly, e.g., for between about 1 to 12 weeks, preferably between 2 to 8 weeks, more preferably between about 3 to 7 weeks, and even more preferably for about 4, 5, or 6 weeks. Factors that may influence the dosage and timing required to effectively treat a subject, include, e.g., the severity of the disease or disorder, formulation, route of delivery, previous treatments, the general health and/or age of the subject, and other diseases present. Moreover, treatment of a subject with a therapeutically effective amount of a compound can include a single treatment or, preferably, can include a series of treatments Animal models can also be used to determine a useful dose, e.g., an initial dose or a regimen.

If a subject is at risk for developing an immunological disorder described herein, the antibody can be administered before the full onset of the immunological disorder, e.g., as a preventative measure. The duration of such preventative treatment can be a single dosage of the antibody or the treatment may continue (e.g., multiple dosages). For example, a subject at risk for the disorder or who has a predisposition for the disorder may be treated with the antibody for days, weeks, months, or even years so as to prevent the disorder from occurring or fulminating.

A pharmaceutical composition may include a “therapeutically effective amount” of an agent described herein. Such effective amounts can be determined based on the effect of the administered agent, or the combinatorial effect of agents if more than one agent is used. A therapeutically effective amount of an agent may also vary according to factors such as the disease state, age, sex, and weight of the individual, and the ability of the compound to elicit a desired response in the individual, e.g., amelioration of at least one disorder parameter or amelioration of at least one symptom of the disorder. A therapeutically effective amount is also one in which any toxic or detrimental effects of the composition are outweighed by the therapeutically beneficial effects.

### Devices and Kits for Therapy

Pharmaceutical compositions that include the anti-TIM-1 antibody can be administered with a medical device. The device can designed with features such as portability, room temperature storage, and ease of use so that it can be used in emergency situations, e.g., by an untrained subject or by emergency personnel in the field, removed from medical facilities and other medical equipment. The device can include, e.g., one or more housings for storing pharmaceutical preparations that include anti-TIM-1 antibody, and can be configured to deliver one or more unit doses of the antibody. The device can be further configured to administer a second agent, e.g., a chemo therapeutic agent, either as a single pharmaceutical composition that also includes the anti-TIM-1 antibody or as two separate pharmaceutical compositions.

The pharmaceutical composition may be administered with a syringe. The pharmaceutical composition can also be administered with a needleless hypodermic injection device, such as the devices disclosed in U.S. Pat. No. 5,399,163; 5,383,851; 5,312,335; 5,064,413; 4,941,880; 4,790,824; or U.S. Pat. No. 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other devices, implants, delivery systems, and modules are also known.

An anti-TIM-1 antibody can be provided in a kit. In one embodiment, the kit includes (a) a container that contains a composition that includes anti-TIM-1 antibody, and optionally (b) informational material. The informational material can be descriptive, instructional, marketing or other material that relates to the methods described herein and/or the use of the agents for therapeutic benefit.

In an embodiment, the kit also includes a second agent for treating a disorder described herein. For example, the kit includes a first container that contains a composition that includes the anti-TIM-1 antibody, and a second container that includes the second agent.

The informational material of the kits is not limited in its form. In one embodiment, the informational material can include information about production of the compound, molecular weight of the compound, concentration, date of expiration, batch or production site information, and so forth. In one embodiment, the informational material relates to methods of administering the anti-TIM-1 antibody, e.g., in a suitable dose, dosage form, or mode of administration (e.g., a dose, dosage form, or mode of administration described herein), to treat a subject who has had or who is at risk for an immunological disorder described herein. The information can be provided in a variety of formats, include printed text, computer readable material, video recording, or audio recording, or information that provides a link or address to substantive material, e.g., on the internet.

In addition to the antibody, the composition in the kit can include other ingredients, such as a solvent or buffer, a stabilizer, or a preservative. The antibody can be provided in any form, e.g., liquid, dried or lyophilized form, preferably substantially pure and/or sterile. When the agents are provided in a liquid solution, the liquid solution preferably is an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the anti-TIM-1 antibody and the second agent, e.g., in a desired ratio. For example, the kit includes a plurality of syringes, ampules, foil packets, blister packs, or medical devices, e.g., each containing a single combination unit dose. The containers of the kits can be air tight, waterproof (e.g., impermeable to changes in moisture or evaporation), and/or light-tight.

The kit optionally includes a device suitable for administration of the composition, e.g., a syringe or other suitable delivery device. The device can be provided pre-loaded with one or both of the agents or can be empty, but suitable for loading.

### Diagnostic Uses

Anti-TIM-1 antibodies can be used in a diagnostic method for detecting the presence of TIM-1, in vitro (e.g., a biological sample, such as tissue, biopsy) or in vivo (e.g., in vivo imaging in a subject). For example, human or effectively human anti-TIM-1 antibodies can be administered to a subject to detect TIM-1 within the subject. For example, the antibody can be labeled, e.g., with an MRI detectable label or a radiolabel. The subject can be evaluated using a means for detecting the detectable label. For example, the subject can be scanned to evaluate localization of the antibody within the subject. For example, the subject is imaged, e.g., by NMR or other tomographic means.

Examples of labels useful for diagnostic imaging include radiolabels such as ¹³¹I, ¹¹¹In, ¹²³I, ^{99m}Tc, ³²P, ³³P, ¹²⁵I, ³H, ¹⁴C, and ¹⁸⁸Rh fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes, can also be employed. The protein ligand can be labeled with such reagents using known techniques. For example, see Wensel and Meares (1983) Radioimmunoimaging and Radioimmunotherapy, Elsevier, New York for techniques relating to the radiolabeling of antibodies and Colcher et al. (1986) Meth. Enzymol. 121: 802-816.

The subject can be “imaged” in vivo using known techniques such as radionuclear scanning using e.g., a gamma camera or emission tomography. See e.g., A. R. Bradwell et al., “Developments in Antibody Imaging”, Monoclonal Antibodies for Cancer Detection and Therapy, R. W. Baldwin et al., (eds.), pp 65-85 (Academic Press 1985). Alternatively, a positron emission transaxial tomography scanner, such as designated Pet VI located at Brookhaven National Laboratory, can be used where the radiolabel emits positrons (e.g., ¹¹C, ¹⁸F, ¹⁵O, and ¹³N).

MRI Contrast Agents. Magnetic Resonance Imaging (MRI) uses NMR to visualize internal features of living subject, and is useful for prognosis, diagnosis, treatment, and surgery. MRI can be used without radioactive tracer compounds for obvious benefit. Some MRI techniques are summarized in EPO 502 814 A. Generally, the differences related to relaxation time constants T1 and T2 of water protons in different environments is used to generate an image. However, these differences can be insufficient to provide sharp high resolution images.

The differences in these relaxation time constants can be enhanced by contrast agents. Examples of such contrast agents include a number of magnetic agents, paramagnetic agents (which primarily alter T1) and ferromagnetic or superparamagnetic agents (which primarily alter T2 response). Chelates (e.g., EDTA, DTPA and NTA chelates) can be used to attach (and reduce toxicity) of some paramagnetic substances (e.g., Fe³⁺, Mn²⁺, Gd³⁺). Other agents can be in the form of particles, e.g., less than 10 μm to about 10 nm in diameter). Particles can have ferromagnetic, anti-ferromagnetic or superparamagnetic properties. Particles can include, e.g., magnetite (Fe₃O₄), γ-Fe₂O₃, ferrites, and other magnetic mineral compounds of transition elements. Magnetic particles may include one or more magnetic crystals with and without nonmagnetic material. The nonmagnetic material can include synthetic or natural polymers (such as sepharose, dextran, dextrin, starch and the like).

The anti-TIM-1 antibodies can also be labeled with an indicating group containing the NMR-active ¹⁹F atom, or a plurality of such atoms inasmuch as (i) substantially all of naturally abundant fluorine atoms are the ¹⁹F isotope and, thus, substantially all fluorine-containing compounds are NMR-active; (ii) many chemically active polyfluorinated compounds such as trifluoracetic anhydride are commercially available at relatively low cost, and (iii) many fluorinated compounds have been found medically acceptable for use in humans such as the perfluorinated polyethers utilized to carry oxygen as hemoglobin replacements. After permitting such time for incubation, a whole body MRI is carried out using an apparatus such as one of those described by Pykett (1982) Scientific American, 246:78-88 to locate and image TIM-1 distribution.

In another aspect, the disclosure provides a method for detecting the presence of TIM-1 in a sample in vitro (e.g., a biological sample, such as serum, plasma, tissue, biopsy). The subject method can be used to diagnose a disorder, e.g., an immunological disorder (e.g., asthma) or a renal disorder (e.g., acute kidney injury, chronic kidney disease, or renal cancer). The method includes: (i) contacting the sample or a control sample with the anti-TIM-1 antibody; and (ii) evaluating the sample for the presence of TIM-1, e.g., by detecting formation of a complex between the anti-TIM-1 antibody and TIM-1, or by detecting the presence of the antibody or TIM-1. For example, the antibody can be immobilized, e.g., on a support, and retention of the antigen on the support is detected, and/or vice versa. A control sample can be included. A statistically significant change in the formation of the complex in the sample relative to the control sample can be indicative of the presence of TIM-1 in the sample. Generally, an anti-TIM-1 antibody can be used in applications that include fluorescence polarization, microscopy, ELISA, centrifugation, chromatography, and cell sorting (e.g., fluorescence activated cell sorting).

The following are examples of the practice of the invention. They are not to be construed as limiting the scope of the invention in any way.

### EXAMPLES

### Example 1

### Monoclonal Antibody ARD5 Binds to a Novel Epitope on the Human TIM-1 Protein

The cDNA corresponding to the long form (364 amino acids) of the human TIM-1 protein was amplified from 769-P human renal adenocarcinoma cell line mRNA using the oligonucleotide primers KID-309 (5′-TAGCGGCCGCAGGCTGATCCCATAATG-3; SEQ ID NO:8) and KID-311 (5′-TAGCGGCCGCTTTCCAGGGACTATTCTC-3; SEQ ID NO:9). Recombinant soluble forms of human TIM-1 protein were made in which the extracellular domain of human TIM-1 (residues 1-290) was attached to the Fc portion of human IgG1 and cloned into pEAG347, a mammalian expression plasmid. Stable CHO cell lines expressing TIM-1-Ig were selected, adapted in suspension, and grown in fermenters. A shorter soluble form of TIM-1 was made in which the Ig-like domain (residues 1-135) was fused to the Fc portion of human IgG1 (hTIM1-Δ mucin-Ig), removing most of the mucin domain, using the oligonucleotide primers SC1-793 (5′-GCGGCCGCTCTAGAATGCATCCTCAAGTGGTCATCTT-3; SEQ ID NO:10) and SC1-794 (5′-ACTAGTGTCGACGGGTGGCACAATCTCCAATGATA-3; SEQ ID NO:11), then cloned in-frame with a human IgG1-Fc coding sequence into vector pv90. This fusion protein was transiently expressed in COS-7 cells. TIM-1-Ig fusion proteins were purified from conditioned media by chromatography on protein A-Sepharose.

Monoclonal antibodies against human TIM-1 were generated in RBF mice immunized with human TIM1-Ig. After standard immunizations, dissociated splenocytes were fused with FL653 myeloma cells and plated by limiting dilution into 96-well tissue culture plates in selection medium. Wells were screened by ELISA assay using 96-well plates coated with human TIM-1-Ig then blocked with BSA. After incubation with the hybridoma supernatant, positive wells were identified using a HRP-coupled secondary antibody that recognizes mouse IgG (Jackson Immunoresearch). A subclone of the monoclonal antibody ARD5 was selected for further study.

Previous studies established that ARD5 is an anti-human TIM-1 monoclonal antibody selective for the IgV domain of human TIM-1 and that it recognizes a non-linear epitope, i.e., an epitope disrupted by denaturation (see Bailly et al. (2002) J. Biol. Chem. 277: 39739-48). To further characterize the monoclonal antibody, cross blocking experiments were performed using a TIM-1 binding ELISA assay. Labeled monoclonal antibodies were tested for binding to human TIM-1-Ig in the presence of excess unlabeled monoclonal antibodies. In this manner, a panel of 23 different anti-TIM-1 monoclonal antibodies was tested in all possible labeled and unlabeled combinations. It was determined which monoclonal antibodies block in only one direction (i.e., A could be blocked by B but B could not be blocked by A), which can be attributed to steric interference, and which monoclonal antibodies blocked in both directions (i.e., A could be blocked by B and B could be blocked by A) which indicated that the two monoclonal antibodies share an epitope recognition site. Using this assay, it was determined that 11 distinct groups of monoclonal antibodies were represented. ARD5 was identified as a monoclonal antibody having a unique epitope that was not shared with any other monoclonal antibody tested.

TIM-1 point mutants were introduced by amplifying a TIM-1 expression plasmid (Origene) with mismatched primers containing appropriate nucleotide changes flanked by 20 base pairs of homologous sequence. PCR was performed using PFU turbo (Stratagene) in S1000 thermal cycler (Bio Rad). Mutations were confirmed by DNA sequencing. HEK 293T cells were transfected with 3 μg wild-type TIM-1, mutant TIM-1, or empty vector expression plasmids using a PEI transfection protocol. 48 hours later cells were lifted with 5 mM EDTA in PBS and washed with PBS containing 5% FBS. Cells were incubated with 0.5 μg of the ARD5, A6G2, AKG7, or A8E5 anti-TIM-1 monoclonal antibody or an IgG2a isotype control in 50 μL, of PBS with 5% FBS for 1 hour on ice. Cells were washed and incubated with an anti-mouse Cy5 (Invitrogen) or FITC (Jackson ImmunoResearch) conjugated secondary for 20 minutes on ice. Cells were washed and expression assessed by measuring % positive cells in FL-4 or FL-1 channels using a FACSCalibur flow cytometer (BD Biosciences).

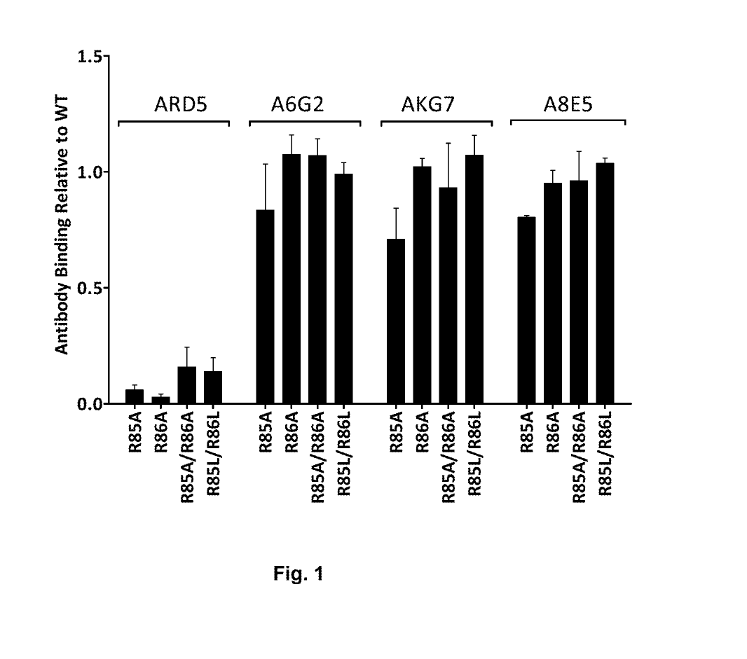

By alanine mutagenesis it was determined that ARD5 binds to an epitope containing the residues Arg85 (R85) and Arg86 (R86) of human TIM-1 (SEQ ID NO:1). Other anti-TIM-1 monoclonal antibodies with distinct TIM-1 binding sites were used as controls for the mutagenesis experiment (FIG. 1). Double mutants R85A/R86A and R85L/R86L were also made and tested for binding to ARD5. No further reduction in binding was observed, indicating that each residue is necessary for ARD5 binding (FIG. 1).
Human TIM-1 amino acid sequence (SEQ ID NO:1). The two arginine residues (Arg85 and Arg86) that contribute to the ARD5 epitope are underlined.

| | |
|---|---|
| 1 | MHPQVVILSL ILHLADSVAG SVKVGGEAGP SVTLPCHYSG AVTSMCWNRG |
| | SCSLFTCQNG |
| 61 | IVWTNGTHVT YRKDTRYKLL GDLSRRDVSL TIENTAVSDS GVYCCRVEHR |
| | GWFNDMKITV |
| 121 | SLEIVPPKVT TTPIVTTVPT VTTVRTSTTV PTTTTVPMTT VPTTTVPTTM |
| | SIPTTTTVLT |
| 181 | TMTVSTTTSV PTTTSIPTTT SVPVTTTVST FVPPMPLPRQ NHEPVATSPS |
| | SPQPAETHPT |
| 241 | TLQGAIRREP TSSPLYSYTT DGNDTVTESS DGLWNNNQTQ LFLEHSLLTA |
| | NTTKGIYAGV |
| 301 | CISVLVLLAL LGVIIAKKYF FKKEVQQLSV SFSSLQIKAL QNAVEKEVQA |
| | EDNIYIENSL |
| 361 | YATD |

By analogy to the mouse TIM-1 sequence and structure, the location of the residues Arg85 and Arg86 was determined to be on the BED face of the protein.
The IgV domain of human TIM1 (SEQ ID NO:2) aligned with the IgV domain of mouse TIM-1 (SEQ ID NO:3). An N-linked glycosylation site and Arg85/Arg86 are underlined in SEQ ID NO:2.

| | | |
|---|---|---|
| HuTIM1 | 1 | MHP-QVVILSLILHLADSVAGSVKVGGEAGPSVT |
| | | LPCHYS |
| MoTIM1 | 1 | MNQIQVFISGLILLLPGTVDSYVEVKGVVGHPVT |
| | | LPCTYS |
| HuTIM1 | 40 | G--AVTSMCWNRGSCSLFTCQNGIVWTNGTHVTY |
| | | RKDTRY |
| MoTIM1 | 41 | TYRGITTTCWGRGQCPSSACQNTLIWTNGHRVTY |
| | | QKSSRY |
| HuTIM1 | 78 | KLLGDLSRRDVSLTIENTAVSDSGVYCCRVEHRG |
| | | WFNDMK |
| MoTIM1 | 81 | NLKGHISEGDVSLTIENSVESDSGLYCCRVEIPG |
| | | WFNDQK |
| HuTIM1 | 118 | ITVSLEIVPPKVTTTPIVTTVPTVTTVRTSTTVP |
| | | TTTTV |
| MoTIM1 | 121 | VTFSLQVKP----------EIPTRPPTRPTTTRP |
| | | TATGR |

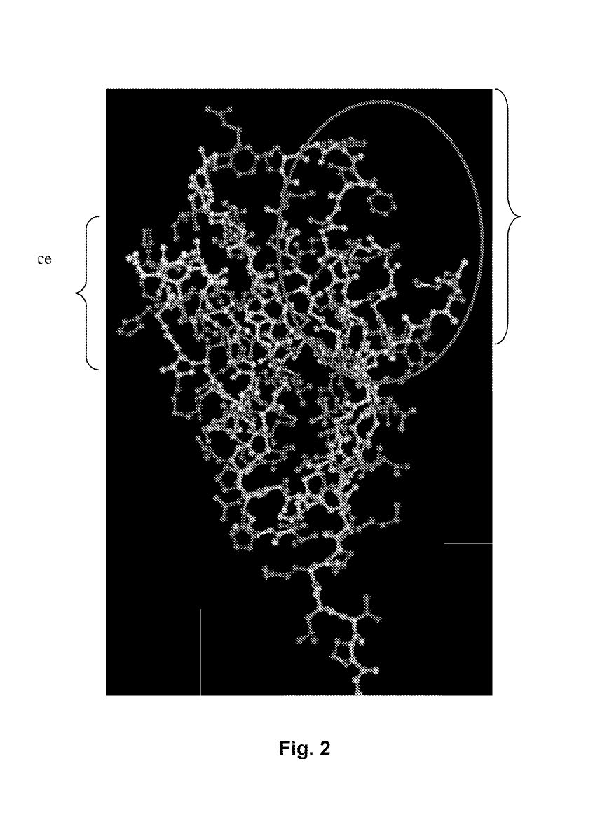

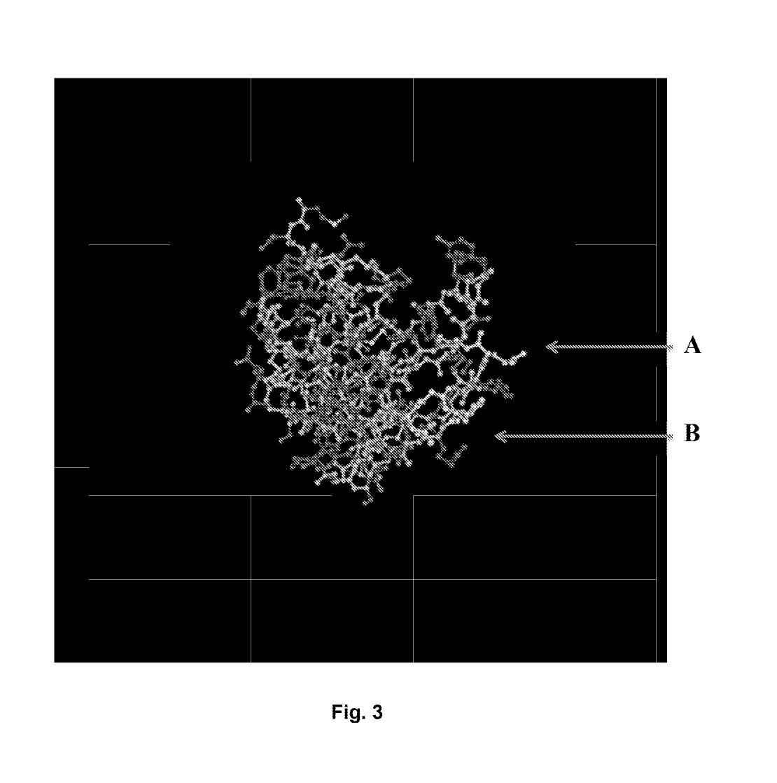

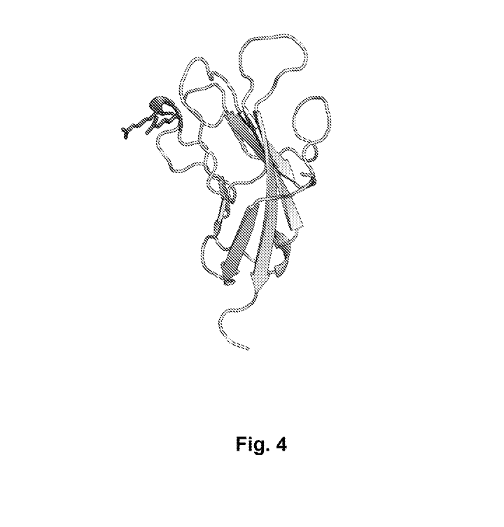

The murine TIM-1 crystal structure was used to model the placement of the Arg85 and Arg86 residues (FIG. 2). The model of mouse TIM-1 was rendered using the Cn3D program (The National Center for Biotechnology Information). These residues lie in plane with an N-linked glycosylation site present in the human TIM-1 (although not the murine TIM-1) protein (FIG. 3). The placement of the epitope was further probed by modeling the human sequence onto the known murine structure (FIG. 4). The model of human TIM-1 was threaded onto the known mouse crystal structure (Santiago et al. 2007) using the Pymol program.

From these analyses, it was concluded that ARD5 binds to human TIM-1 at an epitope that includes amino acid residues Arg85 and Arg86.

### Example 2

### ARD5 Disrupts TIM-1 Binding to Phosphatidylserine

Phosphatidylserine (PS) is a putative TIM-1 ligand. PS-binding assays were performed as described by Sonar et al. (2010) J. Clin. Invest. 120: 2767-81. In brief, 96-well plates (Corning Costar 3590) were coated with a solution of 100 μg/ml PS in methanol and then allowed to dry by evaporation. PS-coated plates were then blocked for 1 hour with 1% BSA in Tris buffer (25 mM Tris, 137 mM NaCl, pH 7.2) before being washed with four times with 0.05% Tween-20 in Tris buffer. The plates were then incubated for one hour with human TIM-1-Fc proteins in 100 μl 1% BSA/Tris buffer. TIM-1-Fc protein was applied across a range of concentrations using 1:3 dilutions starting at 100 μg/ml. After 3 washes with 0.05% Tween-20/Tris buffer, 100 μl/well of a 1:1000 dilution of HRP-conjugated goat anti-human IgG-Fc antibody (Jackson Immunoresearch Laboratories) in 1% BSA/Tris was added for 1 hour. Following 4 additional washes with 0.05% Tween-20/Tris, the plates were developed using 50 μl/well substrate solution (R&D Systems) then stopped by adding 100 μl 2N H2SO4. Plates were read on a microplate reader at 450 nM. Anti-human TIM-1 monoclonal antibody ARD5 was used to compete for binding to PS in Tris buffer containing 1 mM Ca++ and 1 mM Mg++ at a concentration of 10 μg/ml with or without 1 mM EGTA.

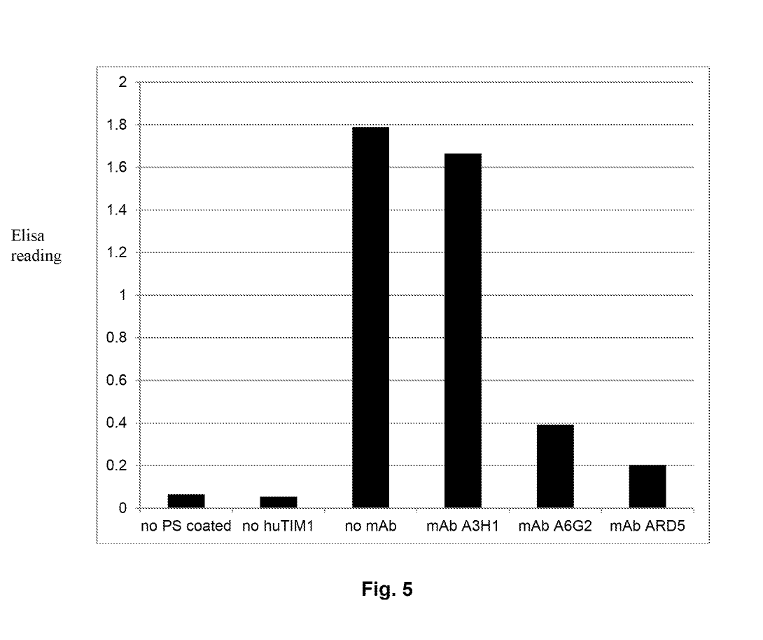

ARD5 blocked the interaction of purified human TIM-1 with PS in the ELISA assay format (FIG. 5). ARD5 blocked the interaction of human TIM-1 with PS to an even greater degree than that observed with monoclonal antibody A6G2, an antibody that recognizes an epitope within the FG/CC′ face of human TIM-1, known to be the site of PS binding (FIG. 5).

### Example 3

### ARD5 Disrupts TIM-1 Binding to Dendritic Cells

The interaction of TIM-1 with dendritic cells is a critical component of immune responses and disease pathology (Sonar et al. (2010) J. Clin. Invest. 120: 2767-81; Feng et al. (2008) J. Allergy Clin. Immunol. 122: 55-61; Degauque et al. (2008) J. Clin. Invest. 118: 735-41; and McIntire et al. (2001) Nat. Immunol. 2: 1109-16).

A flow cytometric assay measuring the binding of highly purified, homogeneous human TIM-1-Ig protein to dendritic cells was used to determine the effect of anti-human TIM-1 monoclonal antibodies on TIM-1/dendritic cell interaction. The dendritic cell-binding assay was performed as described by Sonar et al. (2010) J. Clin. Invest. 120: 2767-81. Briefly, human myeloid dendritic cells were cultured from CD34+ stem cells (Stem Cell Technologies) or were analyzed as CD11c+ dendritic cells from PBMC preparations. Anti-human CD11c monoclonal antibody (BD Biosciences) was used to ensure purity >90% from stem cell cultures and for gating dendritic cell populations in PBMC preparations. TIM-1-Fc fusion proteins were incubated with cells for 1 hour on ice, in the presence or absence of 1 mM EGTA, and in the presence or absence of 10 μg/ml anti-TIM-1 monoclonal antibody ARD5 or an isotype control monoclonal antibody MOPC21 (ATCC). The assay was performed in 1 mM EGTA to eliminate binding to carbohydrate, glycolipid, glycoprotein, and PS, as these interactions require cations. After incubation using fluorochrome-conjugated goat anti-human IgG-Fc (Jackson Immunoresearch Laboratories) cells were fixed in 1% paraformaldehyde and TIM-1-Fc fusion protein binding was analyzed using a flow cytometer (FACScan or LSRII).

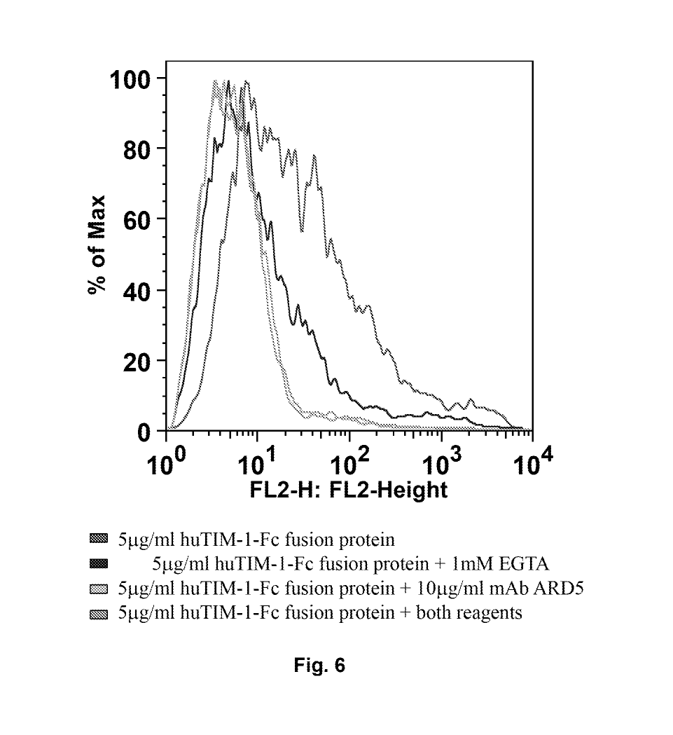

Under these conditions, ARD5 potently and specifically eliminated TIM-1-Ig binding to dendritic cells (FIG. 6). Even in the absence of 1 mM EGTA, ARD5 eliminated essentially all dendritic cell interaction with TIM-1-Ig (FIG. 6). In contrast, monoclonal antibodies recognizing TIM-1 at an epitope at the PS interacting FG/CC′ cleft are less effective in eliminating the TIM-1/dendritic cell interaction.

### Example 4

### ARD5 Binds Human TIM-1 with High Affinity

Three assays were used to assess the affinity of ARD5 for human TIM-1. In the first assay, ARD5 binding to cells expressing TIM-1 was measured by Flow Cytometry. Two cell lines were used. JUN2 cells are a stable human Jurkat TIM-1-expressing cell line (described in Binne et al. (2007) J. Immunol. 178: 4342-50). The human kidney cell line 769-P also constitutively expresses TIM-1. JUN2 cells and 769-P cells were cultured in RPMI/10% FBS. JUN2 cells grow in suspension; 769-P cells are adherent and were gently removed with Accutase (Invitrogen). Cells were resuspended in PBS/5% BSA and incubated with monoclonal antibodies for 1 hour on ice. Following several washes with PBS/5% BSA, the cells were resuspended in the same buffer containing PE-coupled donkey anti-mouse IgG secondary antibody (Jackson Immunoresearch). Following an additional wash in PBS/5% BSA and a final wash in PBS, cell were resuspended in 100 ul PBS and fixed in an equal volume of 2% paraformaldehyde. Monoclonal antibody binding was analyzed using a flow cytometer (FACScan).

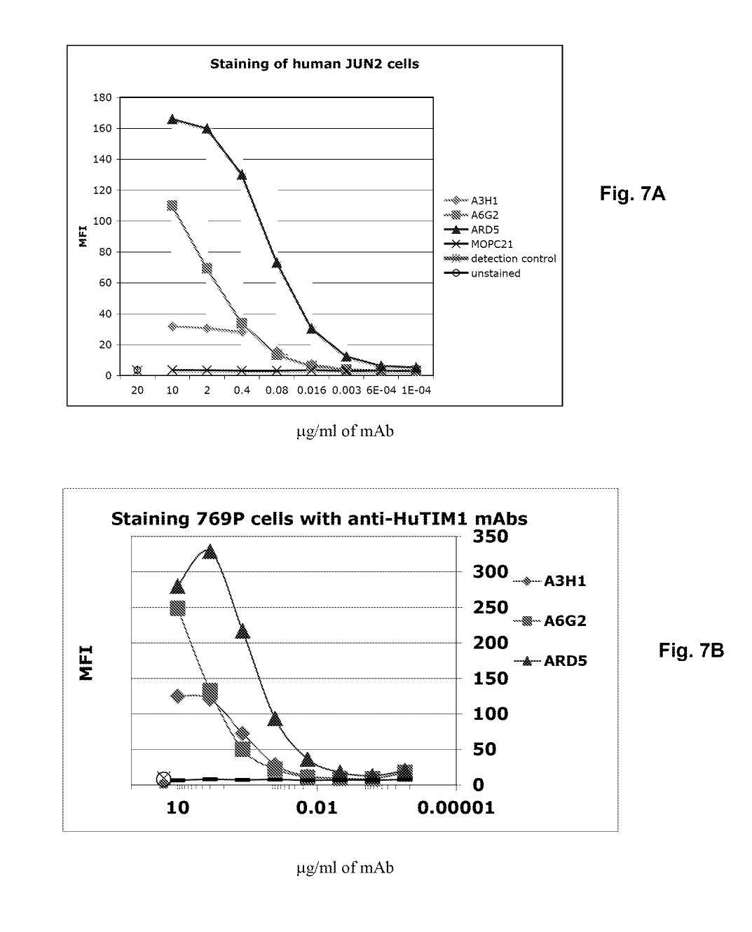

In both flow cytometric assays, the amount of monoclonal antibody added to the cells was titered to derive a binding curve and the maximum mean fluorescence intensity (MFI). In both experiments, ARD5 demonstrated a unique binding profile indicative of a novel mode of high affinity binding. When binding to JUN2 cells stably expressing TIM-1, ARD5 showed detectible binding with as little as 0.6 pg/ml of monoclonal antibody, suggesting a very high affinity, which, per laws of mass action, must be very stable (FIG. 7A). Furthermore, ARD5 reached an MFI 50% greater than that achieved with the monoclonal antibody A6G2 (FIG. 7A). When binding to 769-P cells expressing TIM-1, ARD5 demonstrated detectible binding with as little as 3.2 ng/ml of monoclonal antibody and again reached an MFI 50% greater than that achieved with the monoclonal antibody A6G2 (FIG. 7B). Strikingly, ARD5 binding was saturated at 2 μg/ml, a concentration at which A6G2 signal was sub-optimal.

In the second assay, an ELISA format was used to measure the relative affinity of three anti-TIM-1 monoclonal antibodies for human TIM-1. Titration curves were derived for each antibody and EC50 values were calculated from the curves. Briefly, ELISA plates (Corning Costar) were coated with 0.2 μg/ml human TIM-1-Fc overnight at 4° C. and then blocked for 1 hour at 22° C. with PBS/0.5% casein. Plates were thoroughly washed with PBS/0.1% Tween-20. Anti-human TIM-1 monoclonal antibodies were added across a range of concentrations starting at 10 μg/ml and using 1:3 dilutions, all in PBS/0.5% casein, and allowed to incubate for 1 hour at 22° C. The plates were washed again, and then a 1:1000 dilution of HRP-conjugated goat anti-mouse IgG (H+L; Jackson Immunoresearch Laboratories) was added in PBS/0.5% casein for an additional hour at 22° C. After a final wash, the plates were developed using substrate solution mix (R&D Systems) and stopped using 2N H2SO4. Plates were read at 450 nM using a microplate reader and analyzed using SoftMax Pro (Molecular Devices). Relative EC50 values were determined from the binding curves.

For competition binding assays, each monoclonal antibody was labeled with biotin using Sulfo-NHS-LC-Biotin, as directed by the manufacturer (Pierce). The reaction was run at 10 mg/ml biotin in 260 ul ultrapure H20 for 30 minutes and stopped using 0.1M Tris-HCL, 1M glycine, pH 8.6. The labeled monoclonal antibodies were purified using a 10 ml HiTrap desalting column (GE LifeSciences). Peaks were visualized by FPLC (Akta) as labeled monoclonal antibody, unlabeled monoclonal antibody, and free biotin. Fractions corresponding to the distinct biotin-labeled monoclonal antibody peak were pooled, and concentration was calculated using an area under the curve adjusted OD calculation. The labeled monoclonal antibodies were checked for activity by the above ELISA assay, as visualized using enzymatic readout of streptavidin-coupled HRP (Jackson Immunoresearch Laboratories), which binds avidly to biotin.

ARD5 had the highest EC50 of the antibodies analyzed, as summarized in Table 1. ARD5 had a relative EC50 that was 80.5% greater than that measured for the anti-TIM-1 monoclonal antibodies A6G2 and A3H1 (Table 1).

TABLE 1
Affinity (EC50) for human TIM-1 as measured by ELISA

| monoclonal antibody | affinity (ng/ml) |
|---|---|
| ARD5 | 0.78 |
| A6G2 | 4 |
| A3H1 | 4 |

Surface Plasmon Resonance (Biacore) was used in the final assay. As noted by the equipment manufacturer (www.Biacore.com), the kinetics of an interaction, i.e., the rates of complex formation (kₐ) and dissociation (k_{d}), can be determined from the information in the resulting sensorgram. If binding of the monoclonal antibody occurs as sample passes over a TIM-1-coated sensor surface, the response in the sensorgram increases. When equilibrium is reached, a constant signal is seen. Replacing sample with buffer causes the bound monoclonal antibody to dissociate and the response decreases. Biacore evaluation software generates the values of kₐ and k_{d} by fitting the data to interaction models.

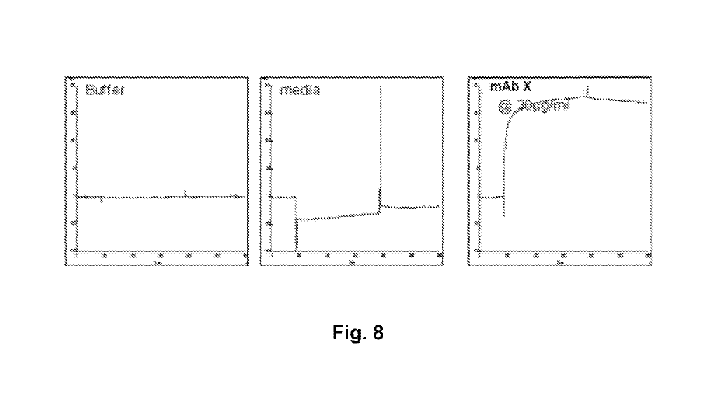

The CMS chip (BIAcore) surface was first activated with N-hydroxy-succinimide/N-ethyl-N′-(3-diethylaminopropyl)-carbodiimide hydrochloride. TIM-1-Fc or isotype control protein was diluted to 30 μl/ml in 10 mM acetic acid (pH 5), and was then injected. The unreacted groups of the chip's dextran matrix were then blocked once with 30 μl and again with 15 μl of ethanolamine-HCl (pH 8.5). This resulted in a surface density of ˜1500 resonance units for the experiments. The chip was regenerated with five 20 μl injections of 1 mM formic acid to establish a reproducible and stable baseline. For the experiment, ARD5 was diluted to 30 μg/ml in diluent buffer. For each run, 100 μl ARD5 or control monoclonal antibody was injected over the surface of the chip. Immediately after each injection, the chip was washed with 300 μl of the diluent buffer and regenerated between experiments with three injections (30, 20, and 10 μl) of 1 mM formic acid. After regeneration, the chip was equilibrated with the diluent buffer. An extremely fast on-rate and slow off-rate of ARD5 binding to human TIM-1 was demonstrated (FIG. 8).

### Example 5

### ARD5 has High Affinity for Non-Human Primate TIM-1

Several methods were used to assess the binding of anti-human TIM-1 monoclonal antibodies to primate TIM-1.

The full-length cDNAs of Cynomolgus monkey (Macaca fascicularis) and Rhesus monkey (Macaca mulatta) TIM-1 were RT-PCR cloned from kidney using oligo dT-primed first strand cDNAs obtained from BioChain Institute and forward primer 5′ CAG AGC TTG GAT CTG AAC GCT GAT CCT ATA ATG 3′ (SEQ ID NO:12) and back primer 5′ GTT CAG TCT TCT GCA GTC ATG GGC GTA AAC TCT 3′ (SEQ ID NO:13), whose sequences were derived from immediate 5′ and 3′ untranslated sequences flanking the predicted open reading frame of the predicted rhesus monkey TIM1 cDNA reported in Genbank accession number XM_{—}01113296. The ˜1.3 kb RT-PCR products were gel-purified and subcloned into Invitrogen's pCRbluntIITOPO vector using their TOPO cloning kit following the manufacturer's recommended protocol. Inserts from multiple independent subclones were sequenced to establish consensus sequences. The cynomolgus monkey full-length TIM-1 cDNA subclone was designated pEAG2172, and the rhesus monkey full-length TIM-1 cDNA subclone was designated pEAG2177. The protein sequences of the TIM-1 IgV domain of these two species were identical. As a result, further experiments were done using the Cynomolgus monkey constructs and proteins.

Shown below is the open reading frame of the full-length Cynomolgus monkey TIM-1 cDNA (SEQ ID NO:14).

| | |
|---|---|
| 1 | ATGCATCCTC AAGTGGTCAT CTTAAGCCTC ATCCTACATC |
| | TGGCAGATTC |
| 51 | TGTAGCTGAT TCTGTAAATG TTGATGGAGT GGCAGGTCTA |
| | CCTATCACAC |
| 101 | TGCCCTGCCG CTACAACGGA GCTATCACAT CCATGTGCTG |
| | GAATAGAGGC |
| 151 | ACATGTTCTG CTTTCTCATG CCCAGATGGC ATTGTCTGGA |
| | CCAATGGAAC |
| 201 | CCACGTCACC TATCGGAAGG AGACACGCTA TAAGCTATTG |
| | GGGAACCTTT |
| 251 | CACGCAGGGA TGTCTCTTTG ACTATAGCAA ATACAGCTGT |
| | GTCTGACAGT |
| 301 | GGCATATATT GTTGCCGTGT TCAGCACAGT GGGTGGTTCA |
| | ATGACATGAA |
| 351 | AATCACCATA TCGTTGAAGA TTGGGCCACC CAGGGTCACA |
| | ACTACTCCAA |
| 401 | TTGTCAGAAC TGTTCGAACA AGCACCACTG TTCCAACGAC |
| | AACGACCCTT |
| 451 | CCAACAACAA CAACTCTTCC AATGACAACG ACAACGACTC |
| | TTCCAACGAC |
| 501 | AACCCTTCCA ATGACGACTC TTCCAATGAC AACGACTCTT |
| | CCAATGACAA |
| 551 | CGACCCTTCC AACGACAACA ACTCTTCCAA CGACAACAAC |
| | TCTTCCAATG |
| 601 | ACAACAACTC TGCCAACGAC AACAACTCTT CCAACGACAA |
| | CGACCCTTCC |
| 651 | AACGACAATG ACTCTTCCAA TGACAACAAC CCTTCCAACG |
| | ACAACAACTC |
| 701 | TGCCAACGAC AACAATGGTC TCTACCTTTG TTCCTCCAAC |
| | GCCATTGCCC |
| 751 | ACGCAGAACC ATGAACCAGC CACTTCACCA TCTTCACCTC |
| | AGCCAGCAGA |
| 801 | AACCCACCCT ATGACACTGC TGGGAGCAAC AAGGACACAA |
| | CCCACCAGCT |
| 851 | CACCATTGTA CTCTTATACA ACAGATGGGA GTGACACCGT |
| | GACAGAGTCT |
| 901 | TCAGATGGCC TTTGGAATAA CAATCAAACT CAATTGTCCC |
| | CAGAACATAG |
| 951 | TCCACAGATG GTCAACACCA CTGAAGGAAT CTATGCTGGA |
| | GTCTGTATTT |
| 1001 | CTGTCTTGGT GCTTCTTGCT GTTTTGGGTG TCGTCATTGC |
| | CAAAAAGTAT |
| 1051 | TTCTTCAAAA AGGAGATTCA ACAACTAAGT GTTTCATTTA |
| | GCAGCCATCA |
| 1101 | AATTAAAACT TTGCAAAATG CAGTTAAAAA GGAAGTCCAC |
| | GCAGAAGACA |
| 1151 | ATATCTACAT TGAGAATCAT CTTTATGCCA TGAACCAAGA |
| | CCCAGTGGTG |
| 1201 | CTCTTTGAGA GTTTACGCCC ATGA |

Shown below is the Cynomologus monkey TIM1 protein sequence (SEQ ID NO:15), which is 79.2% identical to the human TIM-1 protein.

| | |
|---|---|
| 1 | MHPQVVILSL ILHLADSVAD SVNVDGVAGL PITLPCRYNG |
| | AITSMCWNRG |
| 51 | TCSAFSCPDG IVWTNGTHVT YRKETRYKLL GNLSRRDVSL |
| | TIANTAVSDS |
| 101 | GIYCCRVQHS GWFNDMKITI SLKIGPPRVT TTPIVRTVRT |
| | STTVPTTTTL |
| 151 | PTTTTLPMTT TTTLPTTTLP MTTLPMTTTL PMTTTLPTTT |
| | TLPTTTTLPM |
| 201 | TTTLPTTTTL PTTTTLPTTM TLPMTTTLPT TTTLPTTTMV |
| | STFVPPTPLP |
| 251 | TQNHEPATSP SSPQPAETHP MTLLGATRTQ PTSSPLYSYT |
| | TDGSDTVTES |
| 301 | SDGLWNNNQT QLSPEHSPQM VNTTEGIYAG VCISVLVLLA |
| | VLGVVIAKKY |
| 351 | FFKKEIQQLS VSFSSHQIKT LQNAVKKEVH AEDNIYIENH |
| | LYAMNQDPVV |
| 401 | LFESLRP* |

The cynomolgus monkey TIM-1 extracellular domain was expressed as an Fc fusion protein, purified, and used in the ELISA format described above for binding monoclonal antibodies to the human TIM-1 protein. The ELISA plates were coated with human or cynomolgus monkey TIM-1-Fc proteins at 2 μg/ml and relative EC50 values were derived (Table 2, ELISA-1). In a second series of experiments, the ELISA plates were coated with a low concentration of human or cynomolgus monkey TIM-1-Fc protein (0.2 μg/ml) to minimize binding avidity effect and thus highlight differences in intrinsic (monovalent) binding affinity. The resulting titration curves were used to derive a relative EC50 for anti-human TIM-1 monoclonal antibody binding to human TIM-1 and cynomolgus monkey TIM-1 (Table 2, ELISA-2).

The low protein coating condition, in which ‘one-arm’ binding of monoclonal antibodies to protein is favored, demonstrates the very high affinity of the ARD5 monoclonal antibody for human TIM-1 and the near equivalence of ARD5 binding to human TIM-1 and cynomolgus monkey TIM-1 proteins. Also, the ARD5 and A6G2 monoclonal antibodies were bound to human or cynomolgus monkey TIM-1-Fc proteins in a Surface Plasmon Resonance assay using the Biacore platform. The results of this assay reveal the K_{D}s for monovalent ARD5 and A6G2 binding to human or cynomolgus monkey TIM-1 (Table 2, SPR). Finally, FAb fragments of the ARD5 and A6G2 monoclonal antibodies were purified and used in the same Surface Plasmon Resonance assay. The K_{D} for monovalent binding to immobilized human TIM-1 was determined to be 32 nM for the FAb of A6G2 and 3 nM for FAb of ARD5.

TABLE 2
ELISA and Biacore Analyses of Anti-Human TIM-1 Monoclonal
Antibodies Binding to Purified Human and Cynomolgus Monkey TIM-1-Fc Proteins

| | hu.TIM1 | cyno.TIM1 | hu.TIM1 | cyno.TIM1 | hu.TIM1 | cyno.TIM1 |
|---|---|---|---|---|---|---|
| | EC50 | EC50 | EC50 | EC50 | K_{D} | K_{D} |
| | (ng/ml) | (ng/ml) | (ng/ml) | (ng/ml) | (nM) | (nM) |
| Name | ELISA-1 | ELISA-1 | ELISA-2 | ELISA-2 | SPR | SPR |
| A6G2 | 4 | 8 | 380 | 3200 | ≦3 | 53 |
| ARD5 | 1 | 2 | 43 | 13 | ≦0.6 | <<50 |

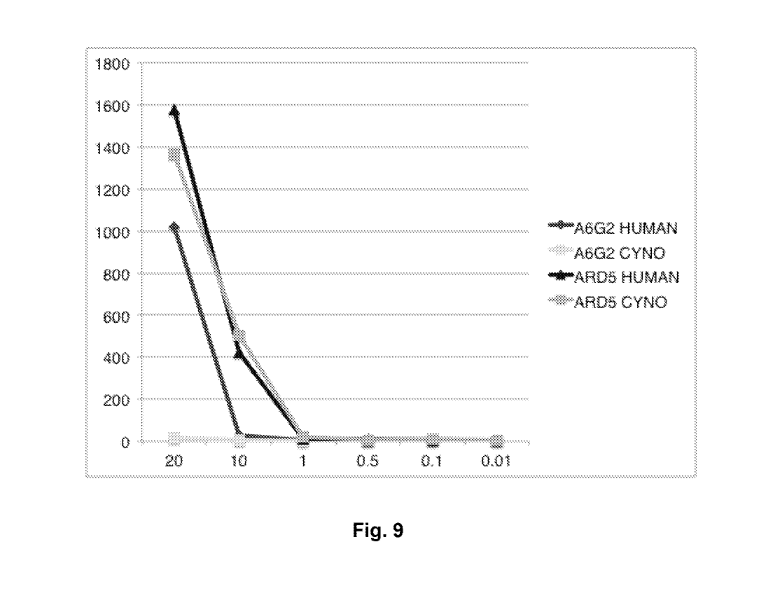

In a second series of experiments, 293E cells transfected with human TIM-1 cDNA or Cynomolgus monkey TIM-1 cDNA were analyzed for anti-human TIM-1 monoclonal antibody binding by flow cytometry. The full-length human and cynomolgus monkey TIM1 cDNAs were engineered to remove extraneous 5′ and 3′ UTRs and to add an identical optimized Kozak sequence, then were subcloned into pNE001, a fully sequence-confirmed pUC-based EBV expression vector derived from the Invitrogen expression vector pCEP4, in which heterologous gene expression is controlled by a CMV-IE promoter and an SV40 polyadenylation signal, but lacking the EBNA gene and the hygromycin resistance gene. TIM-1 expression vectors (human: pEAG2182, and cynomolgus monkey: pEAG2184) were co-transfected into 293E cells at a 1:1 molar ratio with an EBV expression vector carrying an EGFP reporter (pEAG1458). Cells were transfected using Qiagen's Effectene reagent, following the manufacturer's recommended protocol. Cells were used in FACS at 2 days post-transfection, staining with a dilution titration series of murine anti-human TIM1 monoclonal antibodies, (detected with PE-conjugated goat anti-mouse IgG secondary antibody) and gating on green EGFP-positive living cells. ARD5 was found to bind to both human and Cynomolgus monkey overexpressed surface TIM-1 with high affinity. In this assay format, ARD5 bound human and Cynomolgus TIM-1 with overlapping signal intensity, while the monoclonal antibody A6G2 had lower binding to human TIM-1-expressing cells and virtually undetectable binding to the Cynomolgus TIM-1 transfected cells (FIG. 9).

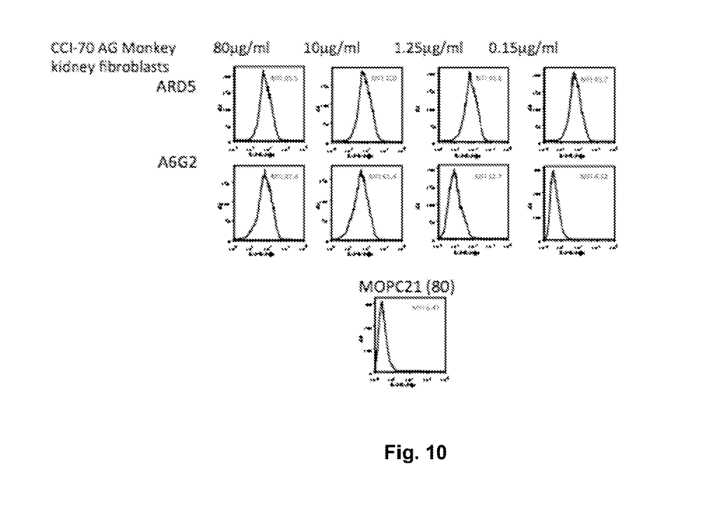

In a similar study, the African Green monkey kidney cell line CCL-70, which constitutively expresses the TIM-1 protein, was tested for binding to ARD5 and A6G2. CCL-70 was cultured in 10 mm tissue culture plates using standard media (RPMI/10% FBS) until they were approximately 80% confluent, then gently removed by incubation with a 1 mM EDTA solution in sterile PBS. Cells were then stained with anti-TIM-1 monoclonal antibodies as described above. Both monoclonal antibodies bound well at concentrations of 80 μg/ml and 10 μg/ml (FIG. 10). While ARD5 exhibited near maximal binding even at 0.15 μg/ml, binding by A6G2 fell off dramatically at concentrations below 10 μg/ml (FIG. 10).

No other monoclonal antibody tested within the anti-human TIM-1 panel demonstrated binding to non-human primate TIM-1 (Cynomolgus monkey or African Green monkey) that resembled the intensity of binding observed with ARD5.

### Example 6

### Cloning of cDNAs Encoding ARD5 Heavy and Light Chain Variable Regions

Total cellular RNA from ARD5 hybridoma cells was prepared using a Qiagen RNeasy mini kit following the manufacturer's recommended protocol. cDNAs encoding the variable regions of the heavy and light chains were cloned by RT-PCR from total cellular RNA, using random hexamers for priming of first strand cDNA. For PCR amplification of the murine immunoglobulin variable domains with signal sequences, a cocktail of degenerate forward primers hybridizing to multiple murine immunoglobulin gene family signal sequences and a single back primer specific for the 5′ end of the murine constant domain. The PCR products were gel-purified and subcloned into Invitrogen's pCR2.1TOPO vector using their TOPO cloning kit following the manufacturer's recommended protocol. Inserts from multiple independent subclones were sequenced to establish a consensus sequence. Assignment to specific subgroups is based upon BLAST analysis using consensus immunoglobulin variable domain sequences from the Kabat database. CDRs are designated using the Kabat definitions (Kabat et al. (1991) Sequences of Proteins of Immunological Interest. 5th Edition, U.S. Dept. of Health and Human Services, U.S. Govt. Printing Office).

Shown below is the ARD5 mature heavy chain variable domain amino acid sequence (SEQ ID NO:4), with CDRs underlined. This is a murine subgroup II(B) heavy chain.

| | |
|---|---|
| 1 | QVQLQQSGAE LVRPGTSVKV SCKASGYVFT NYWIEWIKQR |
| | PGQGLEWIGV |
| 51 | MNPGSGETTY NEKFKGKATL TADKSSSTAY MQLSSLTSVD |
| | SAVYFCARDH |
| 101 | DRDYYAMDYW GQGTSVTVSS |

Shown below is the DNA sequence (SEQ ID NO:5) of the mature ARD5 heavy chain variable domain (from pCN495).

| | |
|---|---|
| 1 | CAGGTACAAC TACAGCAGAG TGGAGCTGAG CTGGTAAGGC |
| | CTGGGACTTC |
| 51 | AGTGAAGGTG TCCTGCAAGG CTTCTGGATA CGTCTTCACT |
| | AATTACTGGA |
| 101 | TAGAGTGGAT AAAGCAGAGG CCTGGACAGG GCCTTGAGTG |
| | GATTGGAGTG |
| 151 | ATGAATCCTG GAAGTGGTGA AACTACCTAC AATGAGAAGT |
| | TCAAGGGCAA |
| 201 | GGCAACACTG ACTGCAGACA AATCCTCCAG CACTGCCTAC |
| | ATGCAGCTCA |
| 251 | GCAGCCTGAC ATCTGTTGAC TCTGCGGTTT ATTTCTGTGC |
| | AAGAGACCAC |
| 301 | GACAGAGATT ACTATGCTAT GGACTACTGG GGTCAGGGAA |
| | CCTCAGTCAC |
| 351 | CGTCTCCTCA |

Shown below is the ARD5 mature light chain variable domain amino acid sequence (SEQ ID NO:6), with CDRs underlined. This is a murine subgroup V kappa light chain.

| | |
|---|---|
| 1 | EIQMTQSPSS MSASLGDTIT ITCQATQDIF KNLNWYQQKP |
| | GKPPSLLIYY |
| 51 | ATELAEGVPS RFSGSGSGSD YSLTISNLES EDFAAYYCLQ |
| | FFEFPFTFGS |
| 101 | GTKLEMK |

Shown below is the DNA sequence (SEQ ID NO:7) of the mature ARD5 light chain variable domain (from pCN496):

| | |
|---|---|
| 1 | GAAATCCAGA TGACCCAGTC TCCATCCTCT ATGTCTGCAT |
| | CTCTGGGAGA |
| 51 | CACAATAACC ATCACTTGCC AGGCAACTCA AGACATTTTT |
| | AAGAATTTAA |
| 101 | ACTGGTATCA GCAGAAACCA GGGAAACCCC CTTCATTGTT |
| | GATCTATTAT |
| 151 | GCAACTGAAC TGGCAGAAGG GGTCCCATCA AGGTTCAGTG |
| | GCAGTGGGTC |
| 201 | TGGGTCAGAC TATTCTCTGA CAATCAGCAA CCTGGAATCT |
| | GAAGATTTTG |
| 251 | CAGCCTATTA CTGTCTACAG TTTTTTGAGT TTCCATTCAC |
| | GTTCGGCTCG |
| 301 | GGGACAAAGT TGGAAATGAA A |

Shown below is the ARD5 mature heavy chain amino acid sequence (SEQ ID NO:18).

| | |
|---|---|
| 1 | QVQLQQSGAE LVRPGTSVKV SCKASGYVFT NYWIEWIKQR |
| | PGQGLEWIGV |
| 51 | MNPGSGETTY NEKFKGKATL TADKSSSTAY MQLSSLTSVD |
| | SAVYFCARDH |
| 101 | DRDYYAMDYW GQGTSVTVSS AKTTPPSVYP LAPGSAAQTN |
| | SMVTLGCLVK |
| 151 | GYFPEPVTVT WNSGSLSSGV HTFPAVLQSD LYTLSSSVTV |
| | PSSTWPSETV |
| 201 | TCNVAHPASS TKVDKKIVPR DCGCKPCICT VPEVSSVFIF |
| | PPKPKDVLTI |
| 251 | TLTPKVTCVV VDISKDDPEV QFSWFVDDVE VHTAQTQPRE |
| | EQFNSTFRSV |
| 301 | SELPIMHQDW LNGKEFKCRV NSAAFPAPIE KTISKTKGRP |
| | KAPQVYTIPP |
| 351 | PKEQMAKDKV SLTCMITDFF PEDITVEWQW NGQPAENYKN |
| | TQPIMDTDGS |
| 401 | YFVYSKLNVQ KSNWEAGNTF TCSVLHEGLH NHHTEKSLSH |
| | SPGK |

Shown below is the ARD5 mature light chain amino acid sequence (SEQ ID NO:19).

| | |
|---|---|
| 1 | EIQMTQSPSS MSASLGDTIT ITCQATQDIF KNLNWYQQKP |
| | GKPPSLLIYY |
| 51 | ATELAEGVPS RFSGSGSGSD YSLTISNLES EDFAAYYCLQ |
| | FFEFPFTFGS |
| 101 | GTKLEMKRAD AAPTVSIFPP SSEQLTSGGA SVVCFLNNFY |
| | PRDINVKWKI |
| 151 | DGSERQNGVL NSWTDQDSKD STYSMSSTLT LTKDEYERHN |
| | SYTCEATHKT |
| 201 | STSPIVKSFN RNEC |

The predicted masses of the deduced mature full-length murine IgG1 heavy and murine kappa light chains are consistent with the masses empirically determined by mass spectroscopy.

### Example 7

### Chimerization of ARD5

cDNAs encoding the murine ARD5 variable regions of the heavy and light chains were used to construct vectors for expression of murine-human chimeras (chARD5) in which the muARD5 variable regions were linked to human IgG1 and kappa constant regions. First, a 0.4 kb PstI-BstEII fragment from pCN495 was ligated to a phosphatased 2.8 kb PstI-BstEII vector fragment from the heavy chain plasmid pLCB7, to add a 5′ NotI site, an optimized Kozak sequence, and a native human signal sequence to the ARD5 murine variable domain, resulting in the cloning intermediate pCN550. For construction of the chimeric heavy chain CHO expression vector, the 0.4 kb NotI-BsmBI fragment from the ARD5 heavy chain variable region subclone pCN550 and the 1.0 kb BsmBI-BamHI fragment from pEAG1325 (a plasmid containing a sequence-confirmed huIgG1 heavy chain constant domain cDNA) were subcloned into the phosphatased 6.0 kb NotI-linearized vector backbone of the expression vector pV90 (in which heterologous gene expression is controlled by a CMV-IE promoter and a human growth hormone polyadenylation signal and which carries a dhfr selectable marker; see U.S. Pat. No. 7,494,805), to produce the expression vector pCN554. The heavy chain cDNA sequence in the resultant plasmid pCN554 was confirmed by DNA sequencing.

For construction of the light chain chimera, the plasmid pCN496 was subjected to PCR with primers 5′ GGG GCG GCC GCA CCA TGA GGG CCC CTG CTC AGT TTC TTG 3′ (SEQ ID NO:16), to introduce a unique NotI site 5′ of the light chain signal sequence, and 5′ CAG TTG GTG CAG CAT CCG TAC GTT TCA TTT CCA A 3′ (SEQ ID NO:17) to introduce a unique BsiWI site immediately downstream of the light chain variable/kappa constant domain junction. Following cleanup on a QIAquick PCR purification spin column, the PCR product was digested with NotI and BsiWI, and the 0.4 kb NotI-BsiWI fragment was gel purified. The 0.4 kb NotI-BsiWI light chain variable domain fragment and the 0.3 kb BsiWI-NotI fragment from the plasmid pEAG1572 (containing a sequence-confirmed human kappa light chain constant domain cDNA) were subcloned into the phosphatased 6.2 kb NotI-linearized vector backbone of the expression vector pV100 (in which heterologous gene expression is controlled by a CMV-IE promoter and a human growth hormone polyadenylation signal and which carries a neomycin selectable marker; see U.S. Pat. No. 7,494,805), to produce plasmid pCN523, the CHO expression vector for chARD5 light chain. The light chain cDNA sequence in plasmid pCN523 was confirmed by DNA sequencing.

Expression of chARD5 was confirmed by transient co-transfection of pCN523 and pCN554 into 293E cells. Transiently transfected cells secreted chARD5 monoclonal antibody into the medium, as confirmed by Western blot of SDS-PAGE gel probed with anti-human IgG (H+L) antibody. FACS staining of TIM-1 expressing cells was performed to demonstrate that chARD5 recapitulated the binding properties of the parent ARD5 murine monoclonal antibody. A stable pool of CHO cells was generated by co-transfection of DG44 cells with plasmids pCN523 and pCN554, followed by dhfr and neo selection.

### Example 8

### ARD5 Exhibits Marked Efficacy in a Humanized Mouse Model of Acute Allergic Asthma

The chimerized ARD5 monoclonal antibody (chARC5) was used in in vivo studies to assess the impact of anti-TIM-1 monoclonal antibody treatment in a humanized mouse model of airway hyperresponsiveness (Sonar et al. (2010) J. Clin. Invest. 120: 2767-81). Briefly, irradiated SCID mice were reconstituted with PBMC from moderate/severe dust-mite-allergic asthmatic patients, followed by multiple immunizations and airway challenges with the dust mite allergen DerP1. Such mice are dust-mite asthmatic humanized mice. This protocol is sufficient to induce human Th2 cytokine expression, human TIM-1 mRNA upregulation on CD4+ T cells, lung tissue inflammation and DerP1-specific human IgE production in the recipient mice. Human cytokine and inflammatory cells are found in bronchial lavage fluid sampled from the challenged mice. Splenic mononuclear cells isolated from the challenged mice proliferate and secrete cytokines in response to ex vivo challenge with DerP1 allergen. Furthermore, challenged mice have a significantly elevated airways hyperresponse to challenge with methacholine. PBMCs isolated from the asthmatic donors directly proliferate and secrete cytokines in response to DerP1 challenge, and do so in a T cell/dendritic cell dependent manner.

Female SCID mice (6-8 weeks old; C.B-17 SCID) were obtained from Harlan Winkelmann (Borchen) and maintained under pathogen-free conditions. All animal experiments were performed in accordance with the appropriate laws and animal care committee guidelines.

Asthmatic patients sensitized to house dust mite allergen were identified by elevated serum D. pteronyssinus-specific IgE antibody titers as measured by fluorescence enzyme immunoassay (Pharmacia CAP System; Pharmacia). Twenty two allergic and asthmatic patients suffering from moderate to severe asthma according to international guidelines (Global Initiative for Asthma) with antibody titers of at least 410 ng/ml for total IgE and at least 20 ng/ml for anti-D. pteronyssinus IgE antibodies were selected as donors and were referred to as asthmatics. Fifteen nonallergic healthy subjects with total serum IgE concentrations of less than 17 ng/ml and anti-D. pteronyssinus IgE of less than 0.8 ng/ml were referred to as nonallergic donors. All blood samples were obtained with written informed consent. Heparinized blood (200-250 ml) was collected from asthmatic and nonallergic donors, and mononuclear cells were purified by Histopaque (Sigma-Aldrich) density gradient centrifugation. Collection of patient and donor samples was approved by the Institutional Review Board at Hochgebirgsklinik, Davos, Switzerland.

Three independent experiments were performed. Each experiment included 6-8 animals per group that received PBMCs from at least 5 asthmatic or nonallergic donors. SCID mice received 2×107 PBMCs i.p. on day 1. Mice also received 100 μl of purified house dust mite extract (Allergopharma Joachim Ganzer), referred to as D. pteronyssinus, with 14 mg/ml of aluminium hydroxide adjuvant (Pierce Biotechnology) on the same day as the cell transfer and on days 7 and 14. All animals were aerosol challenged with 200 μl D. pteronyssinus diluted in 5 ml PBS for 20 minutes on days 16, 18, 20, 23, and 25. Animals also received 100 μl i.p. injections of control IgG1 antibody (MOPC21), anti-human IL-13, anti-human TIM-1 monoclonal antibody ARD5, or anti-human TIM-1 monoclonal antibody A3H1 (a negative control) diluted in 200 μl PBS, or PBS alone.

Splenic mononuclear cells were purified by Histopaque (Sigma-Aldrich) density gradient centrifugation and stimulated with D. pteronyssinus (500 ng/ml) for 72 hours with or without anti-human TIM-1 monoclonal antibody ARD5 (1 μg/ml) or control IgG (1 μg/ml). The supernatants were further processed for cytokine analysis.

Cell proliferation assays were performed using a BrdU labeling and detection kit (Roche). Briefly, 106 mononuclear cells were isolated from mouse spleen or donor PBMCs were incubated in 96-well plates for 24 hours in culture medium alone or with anti-human CD3/CD28 monoclonal antibodies. BrdU was then added to a final concentration of 10 μM/l. After incubation for an additional 24 hours, DNA synthesis was assayed according to the manufacturer's instructions. BrdU-labeled DNA was detected using a luminometer.

Noninvasive measurement of mid-expiratory airflow (EF50) to methacholine was measured 24 hours after the last D. pteronyssinus aerosol challenge using head-out body plethysmography as described previously (43). Dose-response curves of airway reactivity to methacholine was assessed using head-out body plethysmography.

The results are presented as mean values±SEM of 6-8 mice/cohort unless otherwise stated. The Mann-Whitney U test was used to determine the level of significant difference between groups. A P value of 0.05 or lower was considered significant.

Anti-TIM-1 monoclonal antibody treatment of dust-mite asthmatic humanized mice reduces signs and symptoms of asthma (Sonar et al. (2010) J. Clin. Invest. 120: 2767-81). In particular, treatment with monoclonal antibody A6G2, whose epitope includes the ligand-binding cleft present in the human TIM-1 IgV domain, is efficacious in this model.

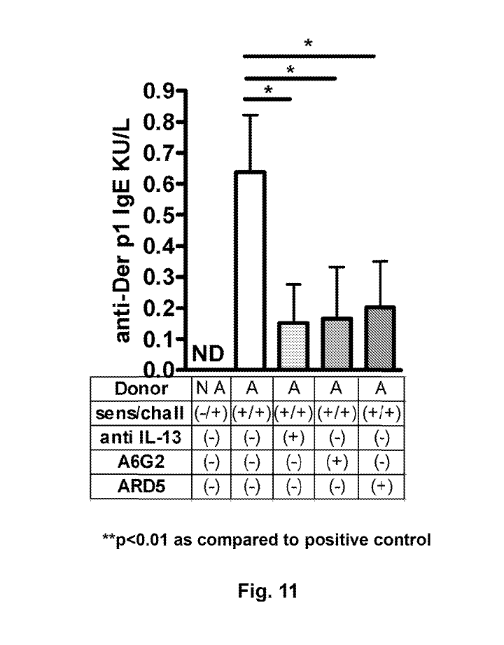

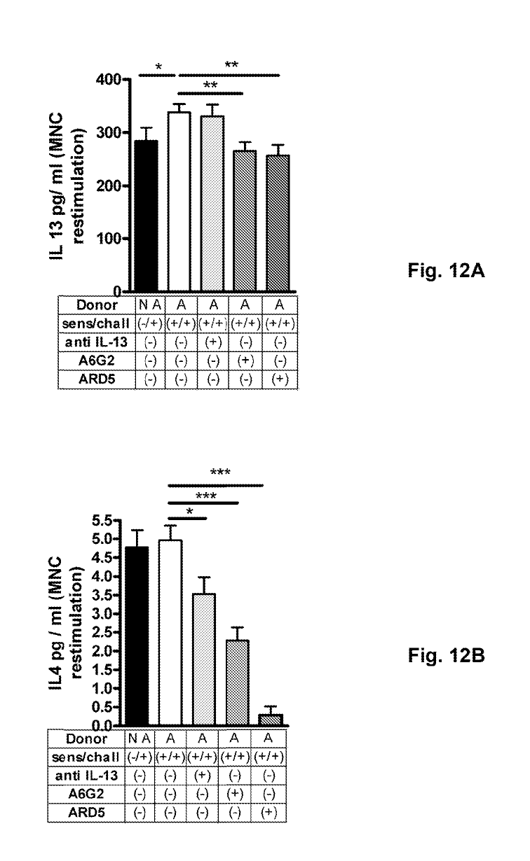

The monoclonal antibody ARD5, which binds to an epitope on the opposite face of the IgV domain as compared to A6G2, was found to markedly reduce signs and symptoms of asthma in the model, to an even greater degree than A6G2. Analysis of dust-mite asthmatic humanized mice treated with ARD5 demonstrated a reduction in DerP1-specific IgE levels to a degree statistically different from the positive control (FIG. 11; positive control is the open rectangle of bar 2; A=asthmatic donor; NA=non-asthmatic donor). Analysis of cytokine production by antigen restimulated splenic mononuclear cells isolated from dust-mite asthmatic humanized mice showed a significantly lower level of IL-13 produced as compared to the positive control and a markedly greater reduction in human IL-4 level in ARD5-treated mice as compared to A6G2-treated mice (FIGS. 12A and 12B; * is p<0.05; ** is p<0.01; *** is p<0.001).

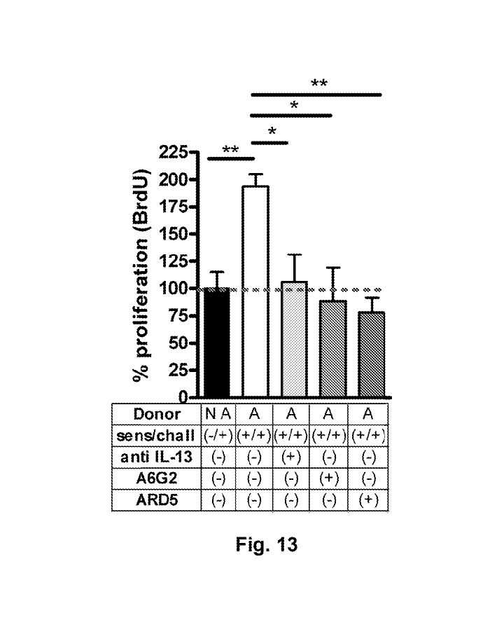

The reduction in cytokine production was accompanied by a statistically greater decrease in cell proliferation in ARD5-treated mice as compared to A6G2-treated mice (FIG. 13; based upon comparisons of the ARD5 and A6G2 treatments to the positive control).

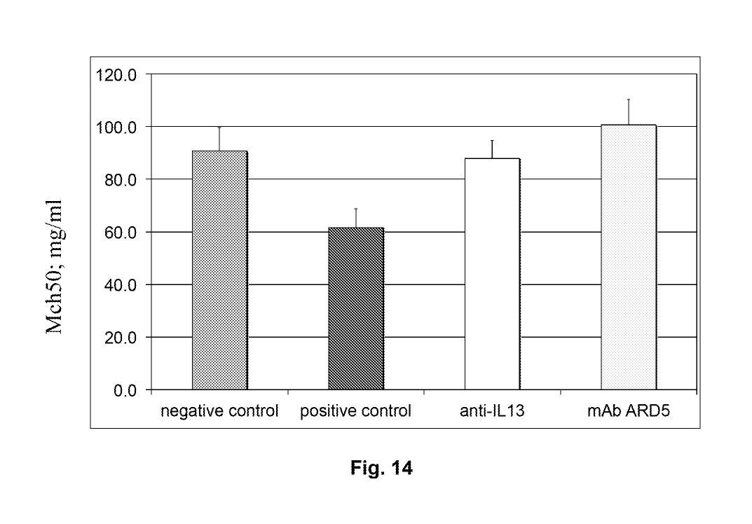

ARD5-treatment was assesed in a dust-mite asthmatic humanized mouse model. Airway hyperresponsiveness was measured using the noninvasive head-out plethysmography method in response to methacholine. The method measured mid-expiratory airflow (EF50) to methacholine 24 hours after the last D. pteronyssinus aerosol challenge using head-out body plethysmography as described in Glaab et al. (2001) Am. J. Physiol. Lung Cell Mol. Physiol. 280(3):L565-L573. The Y-axis provides the dose of methacholine required to induce 50% of the maximal airways hyperresponse. For the positive control (mice reconstituted with PBMC from moderate to severe asthmatic patients) this number is much lower than for the negative control (mice reconstituted with PBMC from normal donors). ARD5 treatment conferred protection equal to or better than the negative control, indicating a higher dose required to induce the hyper-airways response (FIG. 14).

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An isolated antibody or antigen-binding fragment thereof that selectively binds to the polypeptide of SEQ ID NO: 1, when expressed on the surface of a cell, at an epitope that includes arginine amino acid residues at positions 85 and 86 of SEQ ID NO: 1.

2. An isolated antibody or antigen-binding fragment thereof that selectively binds to the polypeptide of SEQ ID NO:1, when expressed on the surface of a cell, and crossblocks binding of the monoclonal antibody ARD5 to SEQ ID NO: 1.

3. An isolated antibody or antigen-binding fragment thereof that selectively binds to the polypeptide of SEQ ID NO: 1, when expressed on the surface of a cell, at the same epitope as the monoclonal antibody ARD5.

4-11. (canceled)

12. An isolated antibody or antigen-binding fragment thereof that (i) selectively binds to the polypeptide of SEQ ID NO: 1, when expressed on the surface of a cell, (ii) comprises a VH domain that is at least 80% identical to the amino acid sequence of SEQ ID NO:4, and (iii) comprises a VL domain that is at least 80% identical to the amino acid sequence of SEQ ID NO:6.

13-14. (canceled)

15. The antibody or antigen-binding fragment thereof of claim 12, wherein (i) the VH domain comprises the amino acid sequence of SEQ ID NO:4, and (ii) the VL domain comprises the amino acid sequence of SEQ ID NO:6.

16-19. (canceled)

20. An isolated antibody or antigen-binding fragment thereof that (i) selectively binds to the polypeptide of SEQ ID NO: 1, when expressed on the surface of a cell, (ii) comprises a VH domain comprising a first heavy chain CDR that is at least 90% identical to CDR-H1 of SEQ ID NO:4, a second heavy chain CDR that is at least 90% identical to CDR-H2 of SEQ ID NO:4, and a third heavy chain CDR that is at least 90% identical to CDR-H3 of SEQ ID NO:4, and (iii) comprises a VL domain comprising a first light chain CDR that is at least 90% identical to CDR-L1 of SEQ ID NO: 6, a second light chain CDR that is at least 90% identical to CDR-L2 of SEQ ID NO:6, and a third light chain CDR that is at least 90% identical to CDR-L3 of SEQ ID NO:6.

21. The antibody or antigen-binding fragment thereof of claim 20, wherein (i) the first heavy chain CDR is identical to CDR-H1 of SEQ ID NO:4, the second heavy chain CDR is identical to CDR-H2 of SEQ ID NO:4, and the third heavy chain CDR is identical to CDR-H3 of SEQ ID NO:4, and (ii) the first light chain CDR is identical to CDR-L1 of SEQ ID NO:6, the second light chain CDR is identical to CDR-L2 of SEQ ID NO: 6, and the third light chain CDR is identical to CDR-L3 of SEQ ID NO: 6.

22. (canceled)

23. The antibody or antigen-binding fragment thereof of claim 1, wherein the antibody is a humanized antibody.

24. The antibody or antigen-binding fragment thereof of claim 1, wherein the antibody is a fully human antibody.

25. The antibody or antigen-binding fragment thereof of claim 1, wherein the antibody is a monoclonal antibody.

26-31. (canceled)

32. An isolated cell that produces the antibody or antigen-binding fragment thereof of claim 1.

33. (canceled)

34. A pharmaceutical composition comprising the antibody or antigen-binding fragment thereof of claim 1 and a pharmaceutically acceptable carrier.

35. A method of inhibiting or reducing binding of TIM-1 to phosphatidylserine, the method comprising contacting a first cell that expresses TIM-1 with an amount of the antibody or antigen-binding fragment thereof of claim 1 effective to inhibit or reduce binding of the first cell to a second cell that contains phosphatidylserine on its cell surface.

36. A method of inhibiting or reducing binding of TIM-1 to a dendritic cell, the method comprising contacting a cell that expresses TIM-1 with an amount of the antibody or antigen-binding fragment thereof of claim 1 effective to inhibit or reduce binding of the cell to a dendritic cell.

37. A method of treating or preventing an inflammatory or autoimmune condition, the method comprising administering to a mammal having an inflammatory or autoimmune condition a pharmaceutical composition comprising a therapeutically effective amount of the antibody or antigen-binding fragment thereof of claim 1.

38. A method of treating or preventing asthma, the method comprising administering to a mammal having asthma a pharmaceutical composition comprising a therapeutically effective amount of the antibody or antigen-binding fragment thereof of claim 1.

39. A method of treating or preventing an atopic disorder, the method comprising administering to a mammal having an atopic disorder a pharmaceutical composition comprising a therapeutically effective amount of the antibody or antigen-binding fragment thereof of claim 1.

40. The method of claim 39, wherein the atopic disorder is atopic dermatitis, contact dermatitis, urticaria, allergic rhinitis, angioedema, latex allergy, or an allergic lung disorder.

41. The method of claim 40, wherein the allergic lung disorder is asthma, allergic bronchopulmonary aspergillosis, or hypersensitivity pneumonitis.

42. The method of claim 37, wherein the mammal is a human.


SEQUENCE LISTING (19 sequences)

SEQ ID NO: 1 - protein, 364 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKGIYAGVCISVLVLLALLGVIIAKKYFFKKEVQQLSVSFSSLQIKALQNAVEKEVQAEDNIYIENSLYATD

SEQ ID NO: 2 - protein, 156 aa, Homo sapiens
MHPQVVILSLILHLADSVAGSVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTV

SEQ ID NO: 3 - protein, 149 aa, Mus sp.
MNQIQVFISGLILLLPGTVDSYVEVKGVVGHPVTLPCTYSTYRGITTTCWGRGQCPSSACQNTLIWTNGHRVTYQKSSRYNLKGHISEGDVSLTIENSVESDSGLYCCRVEIPGWFNDQKVTFSLQVKPEIPTRPPTRPTTTRPTATGR

SEQ ID NO: 4 - protein, 120 aa, Mus sp.
QVQLQQSGAELVRPGTSVKVSCKASGYVFTNYWIEWIKQRPGQGLEWIGVMNPGSGETTYNEKFKGKATLTADKSSSTAYMQLSSLTSVDSAVYFCARDHDRDYYAMDYWGQGTSVTVSS

SEQ ID NO: 5 - DNA, 360 bp, Mus sp.
caggtacaactacagcagagtggagctgagctggtaaggcctgggacttcagtgaaggtgtcctgcaaggcttctggatacgtcttcactaattactggatagagtggataaagcagaggcctggacagggccttgagtggattggagtgatgaatcctggaagtggtgaaactacctacaatgagaagttcaagggcaaggcaacactgactgcagacaaatcctccagcactgcctacatgcagctcagcagcctgacatctgttgactctgcggtttatttctgtgcaagagaccacgacagagattactatgctatggactactggggtcagggaacctcagtcaccgtctcctca

SEQ ID NO: 6 - protein, 107 aa, Mus sp.
EIQMTQSPSSMSASLGDTITITCQATQDIFKNLNWYQQKPGKPPSLLIYYATELAEGVPSRFSGSGSGSDYSLTISNLESEDFAAYYCLQFFEFPFTFGSGTKLEMK

SEQ ID NO: 7 - DNA, 321 bp, Mus sp.
gaaatccagatgacccagtctccatcctctatgtctgcatctctgggagacacaataaccatcacttgccaggcaactcaagacatttttaagaatttaaactggtatcagcagaaaccagggaaacccccttcattgttgatctattatgcaactgaactggcagaaggggtcccatcaaggttcagtggcagtgggtctgggtcagactattctctgacaatcagcaacctggaatctgaagattttgcagcctattactgtctacagttttttgagtttccattcacgttcggctcggggacaaagttggaaatgaaa

SEQ ID NO: 8 - DNA, 27 bp, Artificial Sequence
  Description of Artificial Sequence Synthetic primer
tagcggccgcaggctgatcccataatg

SEQ ID NO: 9 - DNA, 28 bp, Artificial Sequence
  Description of Artificial Sequence Synthetic primer
tagcggccgctttccagggactattctc

SEQ ID NO: 10 - DNA, 37 bp, Artificial Sequence
  Description of Artificial Sequence Synthetic primer
gcggccgctctagaatgcatcctcaagtggtcatctt

SEQ ID NO: 11 - DNA, 35 bp, Artificial Sequence
  Description of Artificial Sequence Synthetic primer
actagtgtcgacgggtggcacaatctccaatgata

SEQ ID NO: 12 - DNA, 33 bp, Artificial Sequence
  Description of Artificial Sequence Synthetic primer
cagagcttggatctgaacgctgatcctataatg

SEQ ID NO: 13 - DNA, 33 bp, Artificial Sequence
  Description of Artificial Sequence Synthetic primer
gttcagtcttctgcagtcatgggcgtaaactct

SEQ ID NO: 14 - DNA, 1224 bp, Macaca fascicularis
atgcatcctcaagtggtcatcttaagcctcatcctacatctggcagattctgtagctgattctgtaaatgttgatggagtggcaggtctacctatcacactgccctgccgctacaacggagctatcacatccatgtgctggaatagaggcacatgttctgctttctcatgcccagatggcattgtctggaccaatggaacccacgtcacctatcggaaggagacacgctataagctattggggaacctttcacgcagggatgtctctttgactatagcaaatacagctgtgtctgacagtggcatatattgttgccgtgttcagcacagtgggtggttcaatgacatgaaaatcaccatatcgttgaagattgggccacccagggtcacaactactccaattgtcagaactgttcgaacaagcaccactgttccaacgacaacgacccttccaacaacaacaactcttccaatgacaacgacaacgactcttccaacgacaacccttccaatgacgactcttccaatgacaacgactcttccaatgacaacgacccttccaacgacaacaactcttccaacgacaacaactcttccaatgacaacaactctgccaacgacaacaactcttccaacgacaacgacccttccaacgacaatgactcttccaatgacaacaacccttccaacgacaacaactctgccaacgacaacaatggtctctacctttgttcctccaacgccattgcccacgcagaaccatgaaccagccacttcaccatcttcacctcagccagcagaaacccaccctatgacactgctgggagcaacaaggacacaacccaccagctcaccattgtactcttatacaacagatgggagtgacaccgtgacagagtcttcagatggcctttggaataacaatcaaactcaattgtccccagaacatagtccacagatggtcaacaccactgaaggaatctatgctggagtctgtatttctgtcttggtgcttcttgctgttttgggtgtcgtcattgccaaaaagtatttcttcaaaaaggagattcaacaactaagtgtttcatttagcagccatcaaattaaaactttgcaaaatgcagttaaaaaggaagtccacgcagaagacaatatctacattgagaatcatctttatgccatgaaccaagacccagtggtgctctttgagagtttacgcccatga

SEQ ID NO: 15 - protein, 407 aa, Macaca fascicularis
MHPQVVILSLILHLADSVADSVNVDGVAGLPITLPCRYNGAITSMCWNRGTCSAFSCPDGIVWTNGTHVTYRKETRYKLLGNLSRRDVSLTIANTAVSDSGIYCCRVQHSGWFNDMKITISLKIGPPRVTTTPIVRTVRTSTTVPTTTTLPTTTTLPMTTTTTLPTTTLPMTTLPMTTTLPMTTTLPTTTTLPTTTTLPMTTTLPTTTTLPTTTTLPTTMTLPMTTTLPTTTTLPTTTMVSTFVPPTPLPTQNHEPATSPSSPQPAETHPMTLLGATRTQPTSSPLYSYTTDGSDTVTESSDGLWNNNQTQLSPEHSPQMVNTTEGIYAGVCISVLVLLAVLGVVIAKKYFFKKEIQQLSVSFSSHQIKTLQNAVKKEVHAEDNIYIENHLYAMNQDPVVLFESLRP

SEQ ID NO: 16 - DNA, 39 bp, Artificial Sequence
  Description of Artificial Sequence Synthetic primer
ggggcggccgcaccatgagggcccctgctcagtttcttg

SEQ ID NO: 17 - DNA, 34 bp, Artificial Sequence
  Description of Artificial Sequence Synthetic primer
cagttggtgcagcatccgtacgtttcatttccaa

SEQ ID NO: 18 - protein, 444 aa, Mus sp.
QVQLQQSGAELVRPGTSVKVSCKASGYVFTNYWIEWIKQRPGQGLEWIGVMNPGSGETTYNEKFKGKATLTADKSSSTAYMQLSSLTSVDSAVYFCARDHDRDYYAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 19 - protein, 214 aa, Mus sp.
EIQMTQSPSSMSASLGDTITITCQATQDIFKNLNWYQQKPGKPPSLLIYYATELAEGVPSRFSGSGSGSDYSLTISNLESEDFAAYYCLQFFEFPFTFGSGTKLEMKRADAAPTVSIFPPSSEQLTSGGASVVCFLNNFYPRDINVKWKIDGSERQNGVLNSWTDQDSKDSTYSMSSTLTLTKDEYERHNSYTCEATHKTSTSPIVKSFNRNEC